(19) United States
(12) Patent Application Publication
(10) Pub. No.: US 2024/0299486 A1
(43) Pub. Date: Sep. 12, 2024

(54) ADDRESSING INJECTION SITE REACTIONS ASSOCIATED WITH THE ADMINISTRATION OF ELAMIPRETIDE

(71) Applicant: STEALTH BIOTHERAPEUTICS INC., Needham, MA (US)

(72) Inventors: Anthony Abbruscato, Needham, MA (US); Alana W. Sullivan, Clayton, NC (US); Laura Kropp, Needham, MA (US)

(73) Assignee: STEALTH BIOTHERAPEUTICS INC., Needham, MA (US)

(21) Appl. No.: 18/666,362

(22) Filed: May 16, 2024

Related U.S. Application Data
(63) Continuation of application No. PCT/US2023/023682, filed on May 26, 2023.
(60) Provisional application No. 63/346,649, filed on May 27, 2022.
(60) Provisional application No. 63/403,149, filed on Sep. 1, 2022.
(60) Provisional application No. 63/455,718, filed on Mar. 30, 2023.

(51) Int. Cl. A61K 38/07; A61K 9/00; A61K 9/06; A61K 31/05; A61K 31/352; A61K 31/37; A61K 31/58

(52) U.S. Cl. CPC A61K 38/07; A61K 9/0019; A61K 9/06; A61K 31/05; A61K 31/352; A61K 31/37; A61K 31/58

(57) ABSTRACT

The present disclosure provides methods for treating, preventing, ameliorating, inhibiting or delaying the onset of injection site reactions associated with the subcutaneous administration of elamipretide, or a pharmaceutically acceptable salt thereof. The methods may involve administration of inhibitors of the MRGPRX2 receptor and/or inhibitors of mast cell degranulation. In some cases, the methods involve administration of an effective amount of a flavonoid, a coumarin, a phenol, or a terpenoid. Such methods may include applying mometasone furoate, tacrolimus, quercetin, diphenhydramine and/or ice to the elamipretide injection site or intended (injection site to treat, prevent, ameliorate, inhibit or delay the onset of said injection site reactions (ISRs). The present disclosure also provides methods for subcutaneously administering a 60 mg dose of elamipretide, or a pharmaceutically acceptable salt thereof, to a subject in need thereof.

### CROSS-REFERENCE TO RELATED APPLICATIONS

This application claims the benefit of and priority to U.S. Provisional Application No. 63/346,649, filed May 27, 2022, U.S. Provisional Application No. 63/403,149, filed Sep. 1, 2022, and U.S. Provisional Application No. 63/455,718, filed Mar. 30, 2023, the contents of each of which are incorporated herein by reference in their entirety for any and all purposes.

### TECHNICAL FIELD

The present technology relates generally to methods for treating, preventing, ameliorating, inhibiting or delaying the onset of injection site reactions (ISRs) associated with the administration of elamipretide, or a pharmaceutically acceptable salt thereof.

### INTRODUCTION

Elamipretide is a cell-permeable tetrapeptide that transiently localizes to the inner mitochondrial membrane, where it reversibly binds to cardiolipin to improve membrane stability, enhance ATP synthesis in several organs, and reduce reactive oxygen species (ROS) production. Elamipretide is being developed for use in the treatment of subjects afflicted with a variety of diseases, disorders, or conditions where mitochondrial dysfunction plays a definitive role in disease etiology, including, heart failure, hypertrophic cardiomyopathy, muscular dystrophy (MD; including Duchene Muscular Dystrophy (DMD and Becker Muscular Dystrophy (BMD)), Friedreich's ataxia (FA), Barth syndrome (BTHS), age-related macular degeneration (AMD), primary mitochondrial myopathy (PMM; including primary mitochondrial disease resulting from nuclear DNA mutations (nPMD)), macular edema, macular degeneration, glaucoma, geographic atrophy, diabetic retinopathy, or retinitis pigmentosa.

### SUMMARY

In some embodiments, the present disclosure provides a method for treating, preventing, ameliorating, inhibiting or delaying the onset of injection site reactions associated with subcutaneous injections of elamipretide, or a pharmaceutically acceptable salt thereof, administered to a subject in need thereof, comprising contacting an elamipretide injection site or intended injection site with an effective amount of an inhibitor of a MRGPRX2 receptor.

In some embodiments, contacting the elamipretide injection site or intended injection site with the inhibitor of the MRGPRX2 receptor treats, prevents, ameliorates, inhibits or delays the onset mast cell degranulation in the subject.

In some embodiments, the method comprises contacting the elamipretide injection site or intended injection site of the subject with an effective amount of a flavonoid, a coumarin, a phenol or a terpenoid as the inhibitor of a MRGPRX2 receptor (See: Finn, D. F. and Walsh, J. J., Twenty-first century mast cell stabilizers, (2013) 170: 23-37). The flavonoid can be luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. The coumarin can be scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). The coumarin can be scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). The terpenoid can be parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the elamipretide injection site or intended injection site is contacted with the flavonoid, the coumarin, the phenol or the terpenoid prior to administration of elamipretide, or a pharmaceutically acceptable salt thereof. In some embodiments, the elamipretide injection site or intended injection site is contacted with the flavonoid, the coumarin, the phenol or the terpenoid after administration of elamipretide, or a pharmaceutically acceptable salt thereof. In some embodiments, the elamipretide injection site or intended injection site is contacted with the flavonoid, the coumarin, the phenol or the terpenoid simultaneously, or substantially simultaneously, with administration of elamipretide, or a pharmaceutically acceptable salt thereof.

In some embodiments, the method comprises contacting the elamipretide injection site or intended injection site of the subject with mometasone furoate, tacrolimus, quercetin, diphenhydramine and/or ice. In some embodiments, mometasone furoate ointment is applied to the elamipretide injection site or intended injection site to thereby contact the elamipretide injection site or intended injection site with mometasone furoate. In some embodiments, tacrolimus ointment or quercetin ointment is applied to the elamipretide injection site or intended injection site to thereby contact the elamipretide injection site or intended injection site with tacrolimus or quercetin. In some embodiments, diphenhydramine or quercetin is administered systemically, optionally via oral administration, to the subject to thereby contact the elamipretide injection site or intended injection site with diphenhydramine or quercetin. In some embodiments, ice is applied to the elamipretide injection site or intended injection site to thereby contact the elamipretide injection site or intended injection site with the ice.

In some embodiments, the elamipretide injection site or intended injection site is contacted with the mometasone furoate, tacrolimus, quercetin, diphenhydramine and/or ice prior to administration of elamipretide, or a pharmaceutically acceptable salt thereof. In some embodiments, the elamipretide injection site or intended injection site is contacted with the mometasone furoate, tacrolimus, quercetin, diphenhydramine and/or ice after administration of elamipretide, or a pharmaceutically acceptable salt thereof. In some embodiments, the elamipretide injection site or intended injection site is contacted with the mometasone furoate, tacrolimus, quercetin, diphenhydramine and/or ice simultaneously or substantially simultaneously with administration of elamipretide, or a pharmaceutically acceptable salt thereof.

In some embodiments, about 5 mg to about 60 mg of elamipretide, or a pharmaceutically acceptable salt thereof, is subcutaneously administered to the elamipretide injection site or intended injection site.

In some embodiments, 60 mg of elamipretide, or a pharmaceutically acceptable salt thereof, is subcutaneously administered at the elamipretide injection site or intended injection site.

In some embodiments, the present disclosure provides a method for treating, preventing, ameliorating, inhibiting or delaying the onset of injection site reactions associated with subcutaneous injections of elamipretide, or a pharmaceutically acceptable salt thereof, administered to a subject in need thereof, comprising contacting an elamipretide injection site or intended injection site with an effective amount of an inhibitor of mast cell degranulation.

In some embodiments, the method comprises contacting the elamipretide injection site or intended injection site of the subject with an effective amount of a flavonoid, a coumarin, a phenol or a terpenoid as the inhibitor of mast cell degranulation. The flavonoid can be luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. The coumarin can be scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). The coumarin can be scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). The terpenoid can be parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the elamipretide injection site or intended injection site is contacted with the flavonoid, the coumarin, the phenol or the terpenoid prior to administration of elamipretide, or a pharmaceutically acceptable salt thereof. In some embodiments, the elamipretide injection site or intended injection site is contacted with the flavonoid, the coumarin, the phenol or the terpenoid after administration of elamipretide, or a pharmaceutically acceptable salt thereof. In some embodiments, the elamipretide injection site or intended injection site is contacted with the flavonoid, the coumarin, the phenol or the terpenoid simultaneously or substantially simultaneously with administration of elamipretide, or a pharmaceutically acceptable salt thereof.

In some embodiments, the method comprises contacting the elamipretide injection site or intended injection site of the subject with mometasone furoate, tacrolimus, quercetin, diphenhydramine and/or ice. In some embodiments, mometasone furoate ointment is applied to the elamipretide injection site or intended injection site to thereby contact the elamipretide injection site or intended injection site with mometasone furoate. In some embodiments, tacrolimus ointment or quercetin ointment is applied to the elamipretide injection site or intended injection site to thereby contact the elamipretide injection site or intended injection site with tacrolimus or quercetin. In some embodiments, diphenhydramine or quercetin is administered systemically, optionally via oral administration, to the subject to thereby contact the elamipretide injection site or intended injection site with diphenhydramine or quercetin. In some embodiments, ice is applied to the elamipretide injection site or intended injection site to thereby contact the elamipretide injection site or intended injection site with the ice.

In some embodiments, the elamipretide injection site or intended injection site is contacted with mometasone furoate, tacrolimus, quercetin, diphenhydramine and/or ice prior to administration of elamipretide, or a pharmaceutically acceptable salt thereof. In some embodiments, the elamipretide injection site or intended injection site is contacted with mometasone furoate, tacrolimus, quercetin, diphenhydramine and/or ice after administration of elamipretide, or a pharmaceutically acceptable salt thereof. In some embodiments, the elamipretide injection site or intended injection site is contacted with mometasone furoate, tacrolimus, quercetin, diphenhydramine and/or ice simultaneously, or substantially simultaneously with, administration of elamipretide, or a pharmaceutically acceptable salt thereof.

In some embodiments, about 5 mg to about 60 mg of elamipretide, or a pharmaceutically acceptable salt thereof, is subcutaneously administered to the elamipretide injection site or intended injection site.

In some embodiments, 60 mg of elamipretide, or a pharmaceutically acceptable salt thereof, is subcutaneously administered to the elamipretide injection site or intended injection site.

In some embodiments, the present disclosure provides a method for treating, preventing, ameliorating, inhibiting or delaying the onset of injection site reactions associated with subcutaneous injections of elamipretide, or a pharmaceutically acceptable salt thereof, administered to a subject in need thereof, comprising contacting an elamipretide injection site or intended injection site with a flavonoid, a coumarin, a phenol or a terpenoid.

The flavonoid can be luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. The coumarin can be scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). The coumarin can be scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). The terpenoid can be parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the elamipretide injection site or intended injection site is contacted with the flavonoid, the coumarin, the phenol or the terpenoid prior to administration of elamipretide, or a pharmaceutically acceptable salt thereof. In some embodiments, the elamipretide injection site or intended injection site is contacted with the flavonoid, the coumarin, the phenol or the terpenoid after administration of elamipretide, or a pharmaceutically acceptable salt thereof. In some embodiments, the elamipretide injection site or intended injection site is contacted with the flavonoid, the coumarin, the phenol or the terpenoid simultaneously, or substantially simultaneously, with administration of elamipretide, or a pharmaceutically acceptable salt thereof.

In some embodiments, about 5 mg to about 60 mg of elamipretide, or a pharmaceutically acceptable salt thereof, is subcutaneously administered to the elamipretide injection site or intended injection site.

In some embodiments, 60 mg of elamipretide, or a pharmaceutically acceptable salt thereof, is subcutaneously administered to the elamipretide injection site or intended injection site.

In some embodiments, the present disclosure provides a method for treating, preventing, ameliorating, inhibiting or delaying the onset of injection site reactions associated with subcutaneous injections of elamipretide, or a pharmaceutically acceptable salt thereof, administered to a subject in need thereof, comprising contacting an elamipretide injection site or intended injection site with an effective amount of mometasone furoate, tacrolimus, quercetin, diphenhydramine and/or ice.

In some embodiments, the elamipretide injection site or intended injection site is contacted with mometasone furoate, tacrolimus, quercetin, diphenhydramine and/or ice prior to administration of elamipretide, or a pharmaceutically acceptable salt thereof. In some embodiments, the elamipretide injection site or intended injection site is contacted with mometasone furoate, tacrolimus, quercetin, diphenhydramine and/or ice after administration of elamipretide, or a pharmaceutically acceptable salt thereof. In some embodiments, the elamipretide injection site or intended injection site is contacted with mometasone furoate, tacrolimus, quercetin, diphenhydramine and/or ice simultaneously, or substantially simultaneously with administration of elamipretide, or a pharmaceutically acceptable salt thereof.

In some embodiments, mometasone furoate ointment is applied to the elamipretide injection site or intended injection site to thereby contact the elamipretide injection site or intended injection site with the mometasone furoate ointment.

In some embodiments, tacrolimus ointment is applied to the elamipretide injection site or intended injection site to thereby contact the elamipretide injection site or intended injection site with tacrolimus ointment or quercetin ointment.

In some embodiments, diphenhydramine or quercetin is administered systemically to the subject to thereby contact the elamipretide injection site or intended injection site with the diphenhydramine.

In some embodiments, ice is applied to the elamipretide injection site or intended injection site to thereby contact the elamipretide injection site or intended injection site with the ice.

In some embodiments, about 5 mg to about 60 mg of elamipretide, or a pharmaceutically acceptable salt thereof, is subcutaneously administered to the elamipretide injection site or intended injection site.

In some embodiments, 60 mg of elamipretide, or a pharmaceutically acceptable salt thereof, is subcutaneously administered to the elamipretide injection site or intended injection site.

In some embodiments, the present disclosure provides a method comprising: a) subcutaneously administering an effective amount of elamipretide, or a pharmaceutically acceptable salt thereof, to a subject; and b) administering to said subject an effective amount of an inhibitor of a MRGPRX2 receptor and/or inhibitor of mast cell degranulation, wherein steps a) and b) can be performed in either order or simultaneously. In some embodiments, step (a) is performed prior to performing step (b). In some embodiments, step (b) is performed prior to performing step (a). In some embodiments, step (a) and step (b) are performed simultaneously or substantially simultaneously.

In some embodiments, the subject is administered an inhibitor of a MRGPRX2 receptor. In some embodiments, the subject is administered an inhibitor of mast cell degranulation.

In some embodiments, the inhibitor of the MRGPRX2 receptor or the inhibitor of mast cell degranulation is a flavonoid, a coumarin, a phenol or a terpenoid. The flavonoid can be luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. The coumarin can be scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). The coumarin can be scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). The terpenoid can be parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, an effective amount of mometasone furoate, tacrolimus, quercetin, diphenhydramine and/or ice is administered to the subject as the inhibitor of the MRGPRX2 receptor or the inhibitor of mast cell degranulation.

In some embodiments, the subject is human.

In some embodiments, from about 20 mg to about 80 mg, from about 30 mg to about 70 mg or from about 40 mg to about 60 mg of elamipretide, or a pharmaceutically acceptable salt thereof, is subcutaneously administered to the subject.

In some embodiments, about 20 mg of elamipretide, or a pharmaceutically acceptable salt thereof, is subcutaneously administered to the subject.

In some embodiments, about 40 mg of elamipretide, or a pharmaceutically acceptable salt thereof, is subcutaneously administered to the subject.

In some embodiments, about 50 mg of elamipretide, or a pharmaceutically acceptable salt thereof, is subcutaneously administered to the subject.

In some embodiments, about 60 mg of elamipretide, or a pharmaceutically acceptable salt thereof, is subcutaneously administered to the subject.

In some embodiments, 60 mg of elamipretide, or a pharmaceutically acceptable salt thereof, is subcutaneously administered to the subject.

In some embodiments, the present disclosure provides a method comprising subcutaneously administering a 60 mg dose of elamipretide, or a pharmaceutically acceptable salt thereof, to a human subject in need thereof to thereby treat, prevent, ameliorate, inhibit or delay the onset of a disease, disorder or condition associated with mitochondrial dysfunction.

In some embodiments, the method further comprises administering to the subject an inhibitor of a MRGPRX2 receptor.

In some embodiments, the method further comprises administering to the subject an inhibitor of mast cell degranulation.

In some embodiments, the inhibitor of the MRGPRX2 receptor or the inhibitor of mast cell degranulation is a flavonoid, a coumarin, a phenol or a terpenoid. The flavonoid can be luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. The coumarin can be scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). The coumarin can be scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). The terpenoid can be parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13 S,16aS,23R,24aS)]-eicosahydro-5H,17H-1,23:11,13-diethano-2H,14H-[1,11]dioxacycloeicosino[2,3-b:12,13-b1]dipyridine).

In some embodiments, mometasone furoate, tacrolimus, quercetin, diphenhydramine and/or ice is/are administered to the subject as the inhibitor of the MRGPRX2 receptor or the inhibitor of mast cell degranulation.

In some embodiments, the subject is human.

In some embodiments, the administration of a 60 mg dose of elamipretide is repeated on a daily basis.

In some embodiments, the subject has been diagnosed with Barth syndrome. In some embodiments, the subject has been diagnosed with Friedreich's ataxia. In some embodiments, the subject has been diagnosed with age-related macular degeneration (AMD). In some embodiments, the subject has been diagnosed with macular edema, macular degeneration, glaucoma, geographic atrophy, diabetic retinopathy, or retinitis pigmentosa. In some embodiments, the subject has been diagnosed with primary mitochondrial myopathy. In some embodiments, the subject has been diagnosed with heart failure. In some embodiments, the subject has been diagnosed with hypertrophic cardiomyopathy. In some embodiments, the subject has been diagnosed with muscular dystrophy (MD); including Duchene Muscular Dystrophy (DMD and Becker Muscular Dystrophy (BMD).

In some embodiments, the subject has been diagnosed with primary mitochondrial disease resulting from nuclear DNA mutation(s) (nPMD).

### BRIEF DESCRIPTION OF THE DRAWINGS

FIG. 1 is an illustration of receptor pathways and common ligands associated with mast cell activation.

FIG. 2 is an illustration of the chemical structure of elamipretide and its known metabolites, M1 and M2.

FIG. 3 is a diagram/illustration showing how and where elamipretide and the various interventions were administered to human subjects in the clinical trial described in Example 1, below.

FIGS. 4A and 4B are representative Fluo-4 fluorescence traces from HEK293 cells stably expressed with MRGPRX2 (FIG. 4B) or its mouse ortholog Mrgprb2 (FIG. 4A). Each trace is from a different cell in the same imaging field. A rise in fluorescence indicates receptor activation.

FIG. 5 is a graphic illustration of the responses of MRGPRX2-expressing HEK293 cells to elamipretide at the indicated concentrations.

FIG. 6A provides representative images of Fluo-4 fluorescence changes in response to elamipretide. FIG. 6B is a bar graph illustration of the average response of wild type or Mrgprb2 knockout primary mouse peritoneal mast cells to 500 micrograms/mL elamipretide. (n=3 mice for each genotype, >200 mast cells counted for each genotype, p<0.01, t-Test, Two-Sample Assuming Unequal Variances).

FIG. 7A provides images of Evans Blue extravasation in wild type and Mrgprb2 knockout mice. Left paws (as illustrated in the image) were injected with 0.5 mg/mL elamipretide, and the right paws were injected with a saline control. Right, quantification of Evans Blue accumulation, measured as specific optical density (OD) per gram of tissue, in the footpads. FIG. 7B is a bar graph illustration of responses were calculated as the OD in elamipretide-injected paws, divided by the OD in saline-injected paws. (n=7 for wild type and n=8 for knockout mice. P<0.01, t-Test, Two-Sample Assuming Unequal Variances).

FIG. 8A is a graphic illustration of the treatment of a human cell line that stably expresses the MRGPRX2 receptor with various concentrations of elamipretide showing that a dose dependent immune response is elicited by the elamipretide. FIG. 8B is a graphic illustration of the treatment of the parent human cell line used in FIG. 8A, but which lacks the MRGPRX2 receptor, with various concentrations of elamipretide thereby demonstrating that there is no immune response in this control cell line lacking the MRGPRX2 receptor. Ionomycin is a membrane-permeable calcium ionophore. It increases intracellular calcium levels in a manner that is not dependent on the presence of the MRGPRX2 receptor.

FIG. 9 is a graphic illustration of the treatment of the human cell line known to express the MRGPRX2 receptor (i.e., the cell line used in FIG. 8A) with Ionomycin (a positive control), Leuprolide (a known agonist of the MRGPRX2 receptor) and DPBS (a negative/vehicle control).

FIG. 10A is a graphic illustration of the treatment of the human cell line known to express the MRGPRX2 receptor (i.e., the cell line used in FIG. 8A) with the metabolite of elamipretide referred to as M1. FIG. 10B is a graphic illustration of the treatment of the human cell line known to express the MRGPRX2 receptor (i.e., the cell line used in FIG. 8A) with the metabolite of elamipretide referred to as M2. The data indicates that, unlike elamipretide, M1 and M2 are not agonists of the MRGPRX2 receptor.

FIG. 11 is a graphic illustration of the treatment of the human cell line known to express the MRGPRX2 receptor (i.e., the cell line used in FIG. 8A) with 100 μM of elamipretide and various concentrations of quercetin, a suspected antagonist of the MRGPRX2 receptor. The data demonstrates that quercetin inhibits activation of MRGPRX2 (or more correctly stated as calcium mobilization that results from MRGPRX2 activation) elicited by elamipretide in a dose-dependent manner.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

In a recently completed phase 1 human clinical trial, injection site reactions were observed as an adverse event that resulted from subcutaneous administration of elamipretide. Similarly, in long term toxicology studies in rats, injection site reactions were observed from subcutaneous administration; some being severe at the higher dose levels administered. Injection site reactions were likewise observed in long term toxicology studies of non-human primates, but these were generally not severe even at the highest dosages tested.

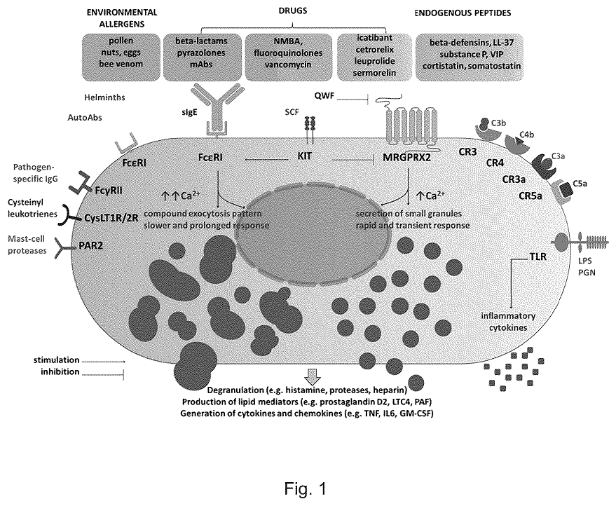

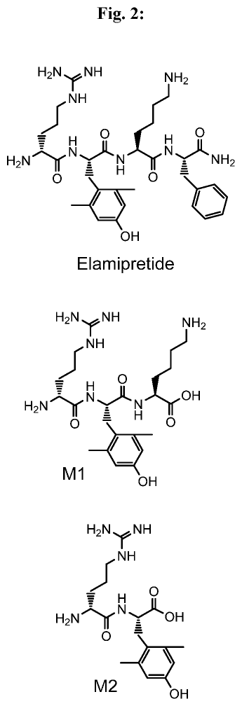

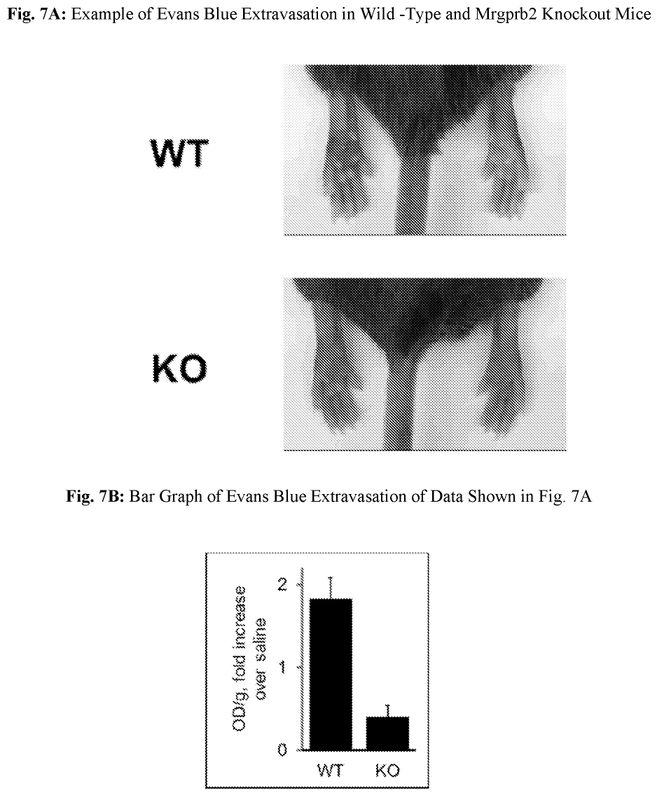

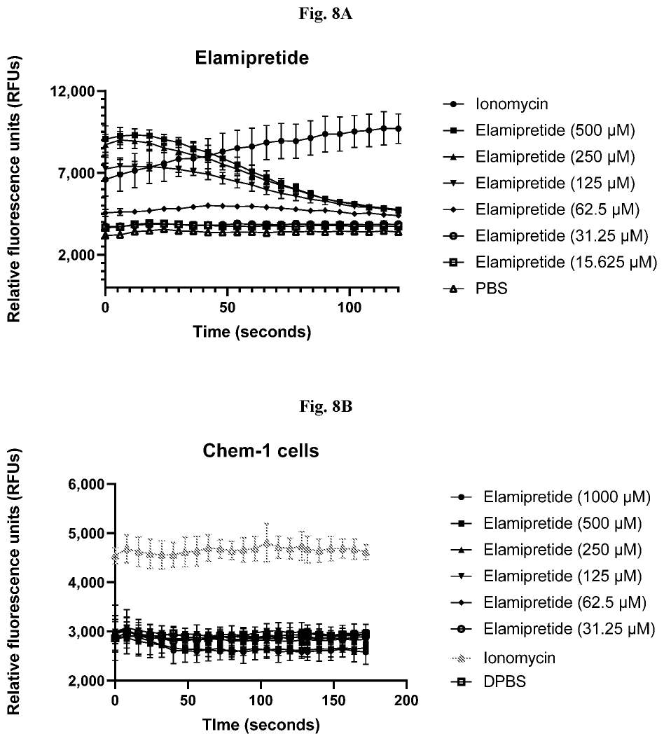

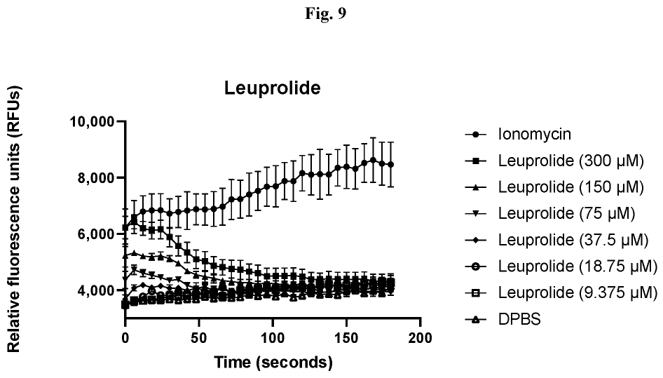

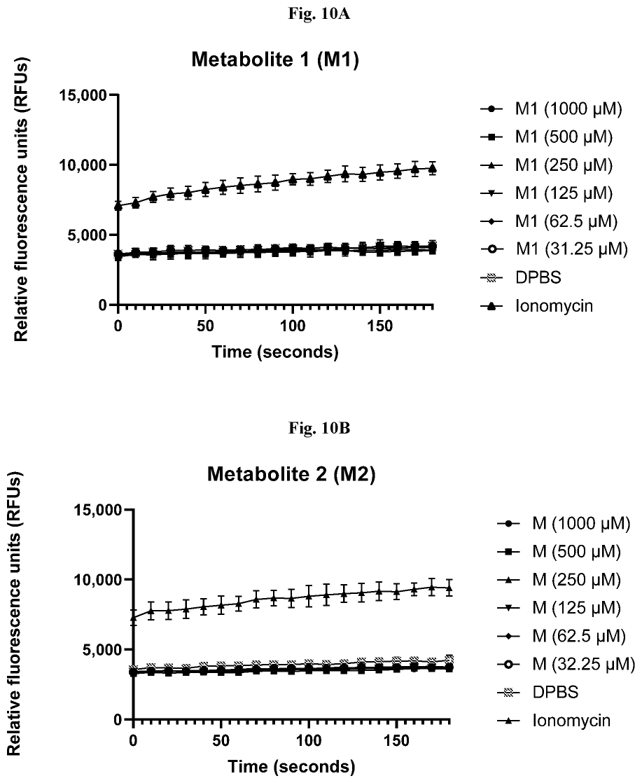

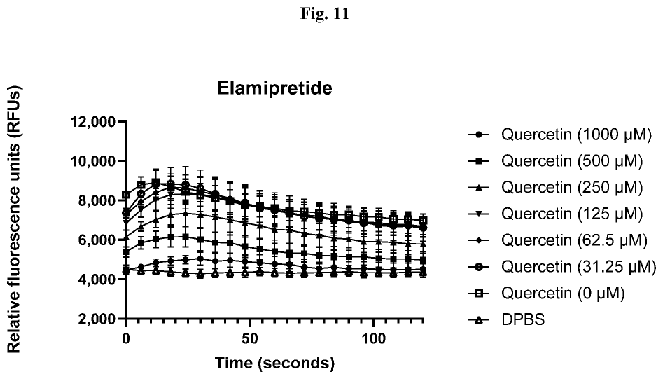

In order to ascertain the basis for these observed injection site reactions, in vitro assays demonstrated that elamipretide activates MRGPRX2, a receptor expressed primarily on skin resident mast cells (FIG. 1: Examples 2 and 3). Further tests have demonstrated that known metabolites of elamipretide (M1 and M2; See: FIG. 2) do not activate this receptor (FIGS. 10A and 10B). Calcium mobilization in response to stimulation with elamipretide is both MRGPRX2-receptor dependent and dose dependent (FIG. 8A, FIG. 8B and FIG. 9). The calcium mobilization in response to stimulation with elamipretide can be inhibited in a dose-dependent manner with quercetin, a suspected antagonist of the MRGPRX2 receptor (FIG. 11). This in vitro data was consistent with animal studies that were conducted (FIGS. 7A and 7B). With this knowledge, a clinical study was initiated to evaluate the potential success of various interventions with respect to injection site reactions caused by SQ administration of elamipretide in human subjects. The results of that study are summarized in Example 1, below.

### I. Definitions

It is to be appreciated that certain aspects, modes, embodiments, variations and features of the technology are described below in various levels of detail in order to provide a substantial understanding of the present application. The definitions of certain terms as used in this specification are provided below. Unless defined otherwise, all technical and scientific terms used herein generally have the same meaning as commonly understood by one of ordinary skill in the art to which this technology belongs.

As used in this specification and the appended embodiments, the singular forms “a”, “an” and “the” include plural references unless the content clearly dictates otherwise. For example, reference to “a cell” includes a combination of two or more cells, and the like.

As used herein, “administering” or the “administration” of an agent (i.e. therapeutic agent) or compound/drug product (including a composition) to a subject includes any route of introducing or delivering to a subject a compound/drug product to perform its intended function. Administration may be carried out by any suitable route, such as oral administration. Administration can be carried out subcutaneously. Administration includes self-administration, the administration by another or administration by use of a device (e.g., an infusion pump).

As used herein, to “ameliorate” or “ameliorating” a disease, disorder or condition refers to results that, in a statistical sample or specific subject, make the occurrence of the disease, disorder or condition (or a sign, symptom or condition thereof) better or more tolerable in a sample or subject administered a therapeutic agent relative to a control sample or subject.

As used herein the terms “carrier” or “pharmaceutically acceptable carrier” refer to a diluent, adjuvant, excipient, or vehicle with which a compound/drug product/composition (including a medicament) is administered or formulated for administration. Non-limiting examples of such pharmaceutically acceptable carriers include liquids, such as water, saline, oils and solids, such as gum acacia, gelatin, starch paste, talc, keratin, colloidal silica, silica particles (nanoparticles or microparticles) urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the phrase “contacting the elamipretide injection site” can also be interpreted as “administering to the elamipretide injection site” wherein the administration can be direct or indirect.

As used herein, the phrase “delaying the onset of” refers to, in a statistical sample, postponing, hindering the occurrence of a disease, disorder or condition, or causing one or more signs, symptoms or conditions to occur more slowly than normal, in a sample or subject administered a therapeutic agent relative to a control sample or subject.

As used herein, the term “effective amount” refers to a quantity of a compound/composition/drug product sufficient to achieve a desired therapeutic and/or prophylactic effect, e.g., an amount that treats, prevents, inhibits, ameliorates, or delays the onset of the disease, disorder or condition, or the physiological signs, symptoms or conditions of the disease or disorder. In the context of therapeutic or prophylactic applications, in some embodiments, the amount of a compound/composition/drug product administered to the subject will depend on the type and severity of the disease and on the characteristics of the individual, such as general health, age, sex, body weight and tolerance to drugs. In some embodiments, it will also depend on the degree, severity and type of disease. The skilled artisan will be able to determine appropriate dosages depending on these and other factors.

The compounds/compositions/drug products can also be administered in combination with one or more additional therapeutic compounds/agents (a so called “co-administration” where, for example, the additional therapeutic agent could be administered simultaneously, sequentially or by separate administration).

As used herein, “elamipretide” refers to the peptide with the amino acid sequence: H-D-Arg-2′,6′Dmt-Lys-Phe-NH₂, where 2′,6′-Dmt is the amino acid 2′,6′-dimethyltyrosine.

Elamipretide has the formula:

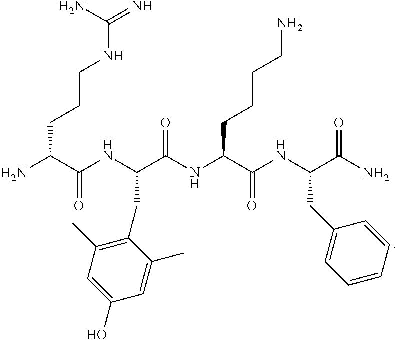 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

Elamipretide is also referred to in the scientific literature as SS-31, bendavia and MTP-131. Elamipretide is typically administered as the pharmaceutically acceptable salt, such as a tris-HCl salt having the formula:

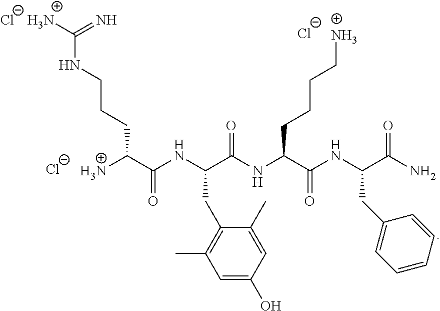 [SMILES: [Cl-] ; Cc1cc(O)cc(C)c1C[C@H](NC(=O)[C@H]([NH3+])CCCNC(=N)[NH3+])C(=O)N[C@@H](CCCC[NH3+])C(=O)N[C@@H](Cc1ccccc1)C(N)=O ; [Cl-] ; [Cl-]]

Whenever the term elamipretide is used herein, its use is intended to also encompass pharmaceutically acceptable salts thereof, unless the context of its use is clearly contradictory to such an interpretation.

As used herein, “inhibit” or “inhibiting” refers to the reduction in injection site reactions as an objectively measurable amount or degree compared to a control. In one embodiment, inhibit or inhibiting refers to the reduction by at least a statistically significant amount compared to a control (or control subject). In one embodiment, inhibit or inhibiting refers to a reduction by at least 5 percent compared to control (or control subject). In various individual embodiments, inhibit or inhibiting refers to a reduction by at least 1, 2, 3, 4, 5, 10, 15, 20, 25, 30, 33, 40, 50, 60, 67, 70, 75, 80, 90, 95, or 99 percent compared to a control (or control subject).

As used herein, “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]+). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

As used herein, “prevention” or “preventing” of a disease, disorder, or condition refers to results that, in a statistical sample, exhibit a reduction in the occurrence of the disease, disorder, or condition in a sample or subject administered a therapeutic agent relative to a control sample or subject. Such prevention is sometimes referred to as a prophylactic treatment.

As used herein, a “subject” refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

As used herein, the terms “treating” or “treatment” refer to therapeutic treatment, wherein the object is to reduce, alleviate or slow down (lessen) a pre-existing disease or disorder, or its related signs, symptoms or conditions. By way of example, but not by way of limitation, a subject is successfully “treated” for a disease if, after receiving an effective amount of the compound/composition/drug product or a pharmaceutically acceptable salt, the subject shows observable and/or measurable reduction in or absence of one or more signs, symptoms or conditions associated with the disease, disorder or condition. It is also to be appreciated that the various modes of treatment of medical conditions as described are intended to mean “substantial,” which includes total alleviation of conditions, signs or symptoms of the disease or disorder, as well as “partial,” where some biologically or medically relevant result is achieved.

### II. Pharmaceutical Compositions, Routes of Administration, and Dosing

While prior studies have shown that subcutaneous (SQ) injections of elamipretide are generally safe and well-tolerated, injection site reactions (ISRs) were reported in 84.4% of subjects receiving SQ elamipretide through clinical trials (Table 1). Injection site reactions were a significant reason for clinical study participants to prematurely depart from active clinical trials. As such, methodologies directed to treating, preventing, ameliorating, inhibiting or delaying the onset of ISRs in subjects could lead to more robust outcomes from clinical trials and improved compliance of subjects under a treatment regime involving SQ administration of elamipretide.

TABLE 1
Summary of Local Treatment Emergent Adverse Events (TEAEs)
Reported in >5% of SQ Elamipretide Treated Subjects
in Multiple-Dose Trials >8 Days

| | Elamipretide Number | | Placebo Number | |
|---|---|---|---|---|
| System Organ Class: | (%) of Subjects | | (%) of Subjects | |
| Preferred Term | (n = 376) | | (n = 199) | |
| Injection site bruising | 45 | (12.0) | 21 | (10.6) |
| Injection site erythema | 178 | (47.3) | 35 | (17.6) |
| Injection site haemorrhage | 22 | (5.9) | 11 | (5.5) |
| Injection site swelling | 52 | (13.8) | 7 | (3.5) |
| Injection site induration | 73 | (19.4) | 8 | (4.0) |
| Injection site mass | 21 | (5.6) | 2 | (1.0) |
| Injection site pain | 84 | (22.3) | 26 | (13.1) |
| Injection site pruritus | 168 | (44.7) | 13 | (6.5) |

| | | | |
|---|---|---|---|
| Injection site urticaria | 47 | (12.5) | 0 |

Elamipretide, or a pharmaceutically acceptable salt thereof, can be administered subcutaneously as a buffered aqueous solution (i.e. preserved, buffered saline) via a needle and syringe. For adults, SQ injections containing 40 mg or 60 mg doses (once per day) have been used with 40 mg being the most common dose. For children, smaller doses can be administered, often 20 mg/day. These are non-limiting examples, as the dose may be varied from patient to patient based on factors such as potency, relative bioavailability, patient body weight, renal impairment, severity of adverse side-effects, toxicity and mode of administration. Elamipretide has been evaluated in the clinic with respect to diseases, disorders and conditions affected by mitochondrial dysfunction. These include, but are not limited to, heart failure, hypertrophic cardiomyopathy, muscular dystrophy (MID; including Duchene Muscular Dystrophy (DMVID and Becker Muscular Dystrophy (BMID)), Friedreich's ataxia (FA), Barth syndrome (BTHS), age-related macular degeneration (AMD) and primary mitochondrial myopathy (PMVM; including primary mitochondrial disease resulting from nuclear DNA mutations (nPMD)).

Mometasone furoate is a corticosteroid used to treat asthma, allergic rhinitis, nasal congestion, nasal polyps, dermatitis, and pruritus. Mometasone furoate is available under the product names: Asmanex®, Dulera®, Elocom™, Elocon®, Nasonex®, Ryaltris®, Sinuva® and Zenhale®. Mometasone furoate can be obtained in a cream/ointment for topical use or as a nasal spray. Mometasone furoate ointment (0.1% w/w) is commonly used for topical administration for relief of the inflammatory and pruritic manifestations of corticosteroid-responsive dermatoses.

Tacrolimus is sold under the trade name Prograf® as an oral formulation. In this form it is generally used with other medicines to help prevent organ rejection in people who have had kidney, liver, heart or lung transplants. Tacrolimus is also sold in ointment form (e.g. 0.03 to 0.1% w/w) for topical administration under the tradenames: Protopic®, Tacrolim® or Fougera®) for use in the treatment of T-cell-mediated diseases such as eczema and psoriasis. It is often used to treat these conditions when more conventional treatments fail. Tacrolimus is an immunosuppressant and belongs to a class of drugs known as topical calcineurin inhibitors (TCIs).

Quercetin is a flavonoid that exhibits antioxidant, anti-inflammatory, antihypertensive, and vasodilator effects/properties. Quercetin is found, inter alia, in onions, grapes, berries, cherries, broccoli and citrus fruit. Quercetin is available in various over-the-counter preparations for oral administration. Quercetin is also available in various over-the-counter preparations for topical administration (i.e., ointment/skin cream forms).

Diphenhydramine (more commonly known as Benadryl®) is an orally administered antihistamine that reduces the effects of natural chemical histamine in the body. Diphenhydramine is an antagonist and acts primarily as an inverse agonist of the histamine H₁ receptor. It is available as a liquid suspension as well as in tablet, caplet and capsule forms; all used for oral administration. Diphenhydramine is used to treat sneezing, runny nose, watery eyes, hives, skin rash, itching, and other cold or allergy symptoms. Diphenhydramine is also used to treat motion sickness, to induce sleep, and to treat certain symptoms of Parkinson's disease. As with other medications, diphenhydramine dosing often depends on the patient's age and patient's body weight.

Icing has traditionally been used to treat such injuries under the premise that it alleviates pain, reduces tissue metabolism, and modifies vascular responses to decrease swelling. Ice may help reduce redness, swelling, and pain in inflammatory-type pimples (e.g., acne).

As stated above, an “effective amount” refers to any amount of the active compound (or compounds; alone or as formulated) that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic (i.e., preventative) or therapeutic treatment regimen can be planned which does not cause substantial unwanted toxicity and yet is effective to treat the particular condition or disease of a particular subject. The effective amount for any particular indication can vary depending on such factors as the disease, disorder or condition being treated, the particular compound or compounds being administered, the size of the subject, or the severity of the disease, disorder or condition. The effective amount may be determined during pre-clinical trials and/or clinical trials by methods familiar to physicians and clinicians. One of ordinary skill in the art can empirically determine the effective amount of a therapeutic agent(s) without necessitating undue experimentation. A maximum dose may be used, that is, the highest safe dose according to some medical judgment. Multiple doses per day may be contemplated to achieve appropriate systemic levels of compounds.

For any therapeutic agent described herein the therapeutically effective amount can, for example, be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

A therapeutic compound/agent (e.g., elamipretide or mometasone furoate or tacrolimus, quercetin or diphenhydramine or ice) disclosed herein can be delivered to the subject in a formulation or medicament (i.e., a pharmaceutical composition). Formulations and medicaments can be prepared by, for example, dissolving or suspending a therapeutic compound/agent disclosed herein in water, a pharmaceutically acceptable carrier, salt, (e.g., NaCl or sodium phosphate), buffering agents, preservatives, compatible carriers, adjuvants, and optionally other therapeutically acceptable ingredients.

The pharmaceutical compositions (e.g., a formulation or medicament) can include a carrier (e.g., a pharmaceutically acceptable carrier), which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Glutathione and other antioxidants can be included to prevent oxidation. In many cases, it will be advantageous to include isotonic agents, for example, sugars (e.g., trehalose), polyalcohols such as mannitol, sorbitol, or sodium chloride in the composition. Prolonged absorption of the injectable compositions can be brought about by including in the composition an agent that delays absorption, for example, aluminum monostearate or gelatin.

Pharmaceutical compositions (e.g., a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and may be preserved against the contaminating action of microorganisms such as bacteria and fungi.

Sterile injectable solutions (e.g., a formulation or medicament) can be prepared by incorporating the active compound (e.g., elamipretide) in the required amount in an appropriate solvent with one or a combination of ingredients enumerated above, as required, followed by filtered sterilization. Generally, dispersions are prepared by incorporating the active compound into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

In addition to the formulations described above, a therapeutic compound/agent (e.g. elamipretide) disclosed herein may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

In some embodiments, the therapeutic agent(s) (e.g., elamipretide) is/are administered as a depot formulation wherein the active therapeutic agent(s) is/are encapsulated by, or disposed within, silica-based microparticles. In some embodiments, the ocular formulation can be injected into the eye, for example as a sol-gel (e.g., a silica sol-gel).

In some embodiments, the ocular formulation is a depot formulation such as a controlled release formulation (see below). Such controlled release formulation may comprise particles, such as microparticles or nanoparticles.

The pharmaceutical compositions also may comprise suitable solid or gel-phase carriers or excipients. Examples of such carriers or excipients include but are not limited to calcium carbonate, calcium phosphate, various sugars, starches, cellulose derivatives, gelatin, silica/silicone and polymers such as polyethylene glycols.

The therapeutic agent(s), including specifically but not limited to a therapeutic compound/agent disclosed herein (e.g. elamipretide), may be provided in particles. Particles as used herein means nanoparticles or microparticles (or in some instances larger particles) which can consist in whole or in part of the therapeutic compound/agent or the other therapeutic agent(s) as described herein. The particles may contain the therapeutic compound(s)/agent(s) (e.g., elamipretide) in a core surrounded by a coating, including, but not limited to, an enteric coating. The therapeutic compound(s)/agent(s) also may be dispersed throughout the particles. The therapeutic compound(s)/agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic compound(s)/agent(s), any of those materials routinely used in the art of pharmacy and medicine, including, but not limited to, erodible, non-erodible, biodegradable, or nonbiodegradable material or combinations thereof. The particles may be microcapsules which contain the therapeutic compound(s)/agent(s) in a solution or in a semi-solid state. The particles may be of virtually any shape.

Both non-biodegradable and biodegradable polymeric materials can be used in the manufacture of particles for delivering the therapeutic compound(s)/agent(s) (e.g., elamipretide). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, polyethylene glycols (PEGs), polyvinylalcohols (PVAs), poly(methyl methacrylates), poly(ethyl methacrylates), poly(butylmethacrylate), poly(isobutyl methacrylate), poly-lactic acid (PLA), poly(lactic-co-glycolic) acid (PLGA), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and poly(8-caprolactone) or mixtures of two or more of the foregoing.

Therapeutic compound(s)/agent(s) (e.g., elamipretide) or mixtures thereof can be formulated in a carrier system. The carrier can be a colloidal system. The carrier or colloidal system can be a liposome, a phospholipid bilayer vehicle. In one embodiment, therapeutic compound(s)/agent(s) or mixtures thereof can be encapsulated in a liposome while maintaining integrity of the therapeutic compound(s)/agent(s) or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound (e.g., elamipretide) or mixtures thereof can be embedded in the polymer matrix, while maintaining integrity of the composition. The polymer can be a microparticle or nanoparticle that encapsulates the therapeutic agent or agents. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In some embodiments, the polymer is poly-lactic acid (PLA), poly lactic/glycolic acid (PLGA) or a mixture thereof. The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the nanoparticles or microparticles can be silica-based or silane-based (See for example: WO2002/080977 entitled: “Biodegradable carrier and method for preparation thereof”).

In some embodiments, the therapeutic compound(s)/agent(s) (e.g. elamipretide) or mixtures thereof are prepared with carriers that will protect the therapeutic compound(s)/agent(s) or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compound(s)/agent(s) (e.g., elamipretide) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom to thereby make it available to the subject. “Delayed release” may or may not involve gradual release of drug over an extended period of time, and thus may or may not be “sustained release.”

Use of a long-term sustained release implant or depot formulation may be particularly suitable for treatment of chronic conditions. The term “implant” and “depot formulation” is intended to include a single composition (such as a mesh) or composition comprising multiple components (e.g., a fibrous mesh constructed from several individual pieces of mesh material) or a plurality of individual compositions where the plurality remains localized and provide the long-term sustained release occurring from the aggregate of the plurality of compositions. “Long-term” release, as used herein, means that the implant or depot formulation is constructed and arranged to deliver therapeutic or prophylactic levels of the active ingredient(s) for at least 2 days. In some embodiments, the implant or depot formulation is constructed and arranged to deliver therapeutic or prophylactic levels of the active ingredient(s) for at least 7 days. In some embodiments, the implant or depot formulation is constructed and arranged to deliver therapeutic or prophylactic levels of the active ingredient(s) for at least 14 days. In some embodiments, the implant or depot formulation is constructed and arranged to deliver therapeutic or prophylactic levels of the active ingredient(s) for at least 30 days. In some embodiments, the implant or depot formulation is constructed and arranged to deliver therapeutic or prophylactic levels of the active ingredient(s) for at least 60 days. In some embodiments, the implant or depot formulation is constructed and arranged to deliver therapeutic or prophylactic levels of the active ingredient for at least 90 days. In some embodiments, the implant or depot formulation is constructed and arranged to deliver therapeutic or prophylactic levels of the active ingredient(s) for at least 180 days. In some embodiments, the implant or depot formulation is constructed and arranged to deliver therapeutic or prophylactic levels of the active ingredient(s) for at least one year. In some embodiments, the implant or depot formulation is constructed and arranged to deliver therapeutic or prophylactic levels of the active ingredient(s) for 15 to 30 days. In some embodiments, the implant or depot formulation is constructed and arranged to deliver therapeutic or prophylactic levels of the active ingredient(s) for 30 to 60 days. In some embodiments, the implant or depot formulation is constructed and arranged to deliver therapeutic or prophylactic levels of the active ingredient(s) for 60 to 90 days. In some embodiments, the implant or depot formulation is constructed and arranged to deliver therapeutic or prophylactic levels of the active ingredient(s) for 90 to 120 days. In some embodiments, the implant or depot formulation is constructed and arranged to deliver therapeutic or prophylactic levels of the active ingredient(s) for 120 to 180 days. In some embodiments, the implant or depot formulation is constructed and arranged to deliver therapeutic or prophylactic levels of the active ingredient(s) for up to one year. In some embodiments, the long-term sustained release implants or depot formulation are well-known to those of ordinary skill in the art and include some of the release systems described above. In some embodiments, such implants or depot formulation can be administered surgically. In some embodiments, such implants or depot formulation can be administered topically or by injection.

### III. Therapeutic Methods

As described herein, an inhibitor of a MRGPRX2 receptor and/or inhibitor of mast cell degranulation may be administered to a subject before, after, or simultaneously with elamipretide. As such, references herein to an “elamipretide injection site” encompass an “intended injection site.”

### a) Inhibition of the MRGPRX2 Receptor

In one aspect, the present disclosure provides a method for treating, preventing, ameliorating, inhibiting or delaying the onset of injection site reactions associated with subcutaneous injections of elamipretide, or a pharmaceutically acceptable salt thereof, administered to a subject in need thereof, comprising contacting an elamipretide injection site or intended injection site with an effective amount of an inhibitor of a MRGPRX2 receptor. In some embodiments, contacting the elamipretide injection site or intended injection site with the inhibitor of the MRGPRX2 receptor treats, prevents, ameliorates, inhibits or delays the onset of mast cell degranulation in the subject. In some embodiments, the contacting is performed by direct application to the surface of the skin at the elamipretide injection site or intended injection site (e.g., by direct application of an ointment or cream). In some embodiments, the contacting is indirect such as by oral administration of the therapeutic agent whereby the inhibitor of the MRGPRX2 receptor is systematically applied thereby reaching many, or all, tissues of the subject, including the injection site or intended injection site.

In some embodiments, contacting the elamipretide injection site or intended injection site with the inhibitor of the MRGPRX2 receptor arrests mast cell degranulation and the resulting immune response at the elamipretide injection site or intended injection site of the subject. In some embodiments, contacting the elamipretide injection site or intended injection site with the inhibitor of the MRGPRX2 receptor treats the elamipretide injection site to thereby arrest mast cell degranulation and the resulting immune response at the elamipretide injection site of the subject. In some embodiments, contacting the elamipretide injection site or intended injection site with the inhibitor of the MRGPRX2 receptor prevents mast cell degranulation and the resulting immune response at the elamipretide injection site of the subject. In some embodiments, contacting the elamipretide injection site or intended injection site with the inhibitor of the MRGPRX2 receptor ameliorates mast cell degranulation and the resulting immune response at the elamipretide injection site of the subject. In some embodiments, contacting the elamipretide injection site or intended injection site with the inhibitor of the MRGPRX2 receptor inhibits mast cell degranulation and the resulting immune response at the elamipretide injection site of the subject. In some embodiments, contacting the elamipretide injection site or intended injection site with the inhibitor of the MRGPRX2 receptor delays the onset of the injection site reaction by arresting mast cell degranulation and resulting immune response at the elamipretide injection site of the subject.

In some embodiments, the elamipretide injection site or intended injection site is contacted with the inhibitor of the MRGPRX2 receptor prior to administration of elamipretide, or a pharmaceutically acceptable salt thereof. In some embodiments, the elamipretide injection site or intended injection site is contacted with the inhibitor of the MRGPRX2 receptor after administration of elamipretide, or a pharmaceutically acceptable salt thereof. In some embodiments, the elamipretide injection site or intended injection site is contacted with the inhibitor of the MRGPRX2 receptor simultaneously with administration of elamipretide, or a pharmaceutically acceptable salt thereof.

In some embodiments, the method comprises contacting the elamipretide injection site or intended injection site with an effective amount of a flavonoid, a coumarin, a phenol or a terpenoid as the inhibitor of the MRGPRX2 receptor. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the elamipretide injection site or intended injection site is contacted with the flavonoid, a coumarin, a phenol or a terpenoid prior to administration of elamipretide, or a pharmaceutically acceptable salt thereof. In some embodiments, the elamipretide injection site or intended injection site is contacted with the flavonoid, a coumarin, a phenol or a terpenoid after administration of elamipretide, or a pharmaceutically acceptable salt thereof. In some embodiments, the elamipretide injection site or intended injection site is contacted with the flavonoid, a coumarin, a phenol or a terpenoid simultaneously with administration of elamipretide, or a pharmaceutically acceptable salt thereof.

In some embodiments, the inhibitor of the MRGPRX2 receptor is mometasone furoate, tacrolimus, quercetin, and/or diphenhydramine. In some embodiments, the inhibitor is ice. Thus, in some embodiments, the method comprises contacting the elamipretide injection site or intended injection site with mometasone furoate, tacrolimus, quercetin, diphenhydramine and/or ice prior to administration of elamipretide, or a pharmaceutically acceptable salt thereof. In some embodiments, the elamipretide injection site or intended injection site is contacted with the mometasone furoate, tacrolimus, quercetin, and/or diphenhydramine and/or ice after administration of elamipretide, or a pharmaceutically acceptable salt thereof. In some embodiments, the elamipretide injection site or intended injection site is contacted with mometasone furoate, tacrolimus, quercetin, and/or diphenhydramine and/or ice simultaneously with administration of elamipretide, or a pharmaceutically acceptable salt thereof.

The administration of the inhibitor of the inhibitor of the MRGPRX2 receptor can be direct (e.g., applied to the area of the skin where the injection of elamipretide is/was administered) or indirect (e.g., where the therapeutic agent is/was administered systematically (e.g., oral or nasal administration) and therefore is applied to the elamipretide injection site by operation of the biological processes of the subject that directs the therapeutic agent to the skin cells in the area of the elamipretide injection site (and elsewhere in the body of the subject). Thus, in some embodiments, mometasone furoate ointment is applied to the elamipretide injection site or intended injection site to thereby contact the elamipretide injection site or intended injection site with mometasone furoate. In some embodiments, tacrolimus ointment or quercetin ointment is applied to the elamipretide injection site or intended injection site to thereby contact the elamipretide injection site with tacrolimus or quercetin. In some embodiments, diphenhydramine or quercetin is administered systemically, optionally via oral administration, to the subject to thereby contact the elamipretide injection site or intended injection site with diphenhydramine or quercetin. In some embodiments, ice is applied to the elamipretide injection site or intended injection site to thereby contact the elamipretide injection site with the ice.

In some embodiments of the method, the elamipretide injection site or intended injection site is contacted with mometasone furoate ointment. In some embodiments of the method, the elamipretide injection site or intended injection site is contacted with tacrolimus ointment. In some embodiments of the method, the elamipretide injection site or intended injection site is contacted with quercetin, either as a topical ointment or cream or indirectly by oral administration of the quercetin. In some embodiments of the method, the elamipretide injection site or intended injection site is contacted with tacrolimus or quercetin.

In some embodiments of the method, the elamipretide injection site or intended injection site is contacted with diphenhydramine, often indirectly/systemically by oral administration of the diphenhydramine. In some embodiments of the method, the elamipretide injection site or intended injection site is contacted with quercetin, indirectly/systemically by oral administration of the quercetin. In some embodiments of the method, the elamipretide injection site or intended injection site is contacted with diphenhydramine or quercetin, indirectly/systemically by oral administration of the diphenhydramine or quercetin. In some embodiments of the method, ice is applied to the elamipretide injection site.

In some embodiments of the aforementioned methods, about 5 mg to about 80 mg of elamipretide, or a pharmaceutically acceptable salt thereof, is subcutaneously administered to the injection site of the subject. In some embodiments of the aforementioned methods, about 10 mg to about 60 mg of elamipretide, or a pharmaceutically acceptable salt thereof, is subcutaneously administered to the injection site of the subject. In some embodiments of the aforementioned methods, about 20 mg to about 60 mg of elamipretide, or a pharmaceutically acceptable salt thereof, is subcutaneously administered to the injection site of the subject. In some embodiments of the aforementioned methods, about 20 mg of elamipretide, or a pharmaceutically acceptable salt thereof, is subcutaneously administered to the injection site of the subject. In some embodiments of the aforementioned methods, about 40 mg of elamipretide, or a pharmaceutically acceptable salt thereof, is subcutaneously administered to the injection site of the subject. In some embodiments of the aforementioned methods, about 60 mg of elamipretide, or a pharmaceutically acceptable salt thereof, is subcutaneously administered to the injection site of the subject. In some embodiments of the aforementioned methods, 60 mg of elamipretide, or a pharmaceutically acceptable salt thereof, is subcutaneously administered to the injection site of the subject.

### b) Inhibition of the Mast Cell Degranulation

In one aspect, the present disclosure provides a method for treating, preventing, ameliorating, inhibiting or delaying the onset of injection site reactions associated with subcutaneous injections of elamipretide, or a pharmaceutically acceptable salt thereof, administered to a subject in need thereof, comprising contacting an elamipretide injection site or intended injection site with an effective amount of an inhibitor of mast cell degranulation. In some embodiments, contacting the elamipretide injection site or intended injection site with the inhibitor of mast cell degranulation treats, prevents, ameliorates, inhibits or delays the onset mast cell degranulation in the subject. In some embodiments, the contacting is performed by direct application to the surface of the skin at the elamipretide injection site or intended injection site (e.g., by direct application of an ointment or cream). In some embodiments, the contacting is indirect such as by oral administration of the therapeutic agent whereby the inhibitor of the mast cell degranulation is systematically applied thereby reaching many, or all, tissues of the subject, including the elamipretide injection site or intended injection site.

In some embodiments, contacting the elamipretide injection site or intended injection site with the inhibitor of mast cell degranulation arrests mast cell degranulation and the resulting immune response at the elamipretide injection site of the subject. In some embodiments, contacting the elamipretide injection site or intended injection site with the inhibitor of mast cell degranulation treats the elamipretide injection site to thereby arrest mast cell degranulation and the resulting immune response at the elamipretide injection site of the subject. In some embodiments, contacting the elamipretide injection site or intended injection site with the inhibitor of mast cell degranulation prevents mast cell degranulation and the resulting immune response at the elamipretide injection site of the subject. In some embodiments, contacting the elamipretide injection site or intended injection site with the inhibitor of mast cell degranulation ameliorates mast cell degranulation and the resulting immune response at the elamipretide injection site of the subject. In some embodiments, contacting the elamipretide injection site or intended injection site with the inhibitor of mast cell degranulation inhibits mast cell degranulation and the resulting immune response at the elamipretide injection site of the subject. In some embodiments, contacting the elamipretide injection site or intended injection site with the inhibitor of mast cell degranulation delays the onset of the injection site reaction by arresting mast cell degranulation and resulting immune response at the elamipretide injection site of the subject.

In some embodiments, contacting the elamipretide injection site or intended injection site with the inhibitor or mast cell degranulation can be can be direct (e.g., applied to the area of the skin where the injection is/was administered) or indirect (e.g., where the inhibitor or mast cell degranulation is administered systematically (e.g., oral or nasal administration) and therefore is applied to the elamipretide injection site or intended injection site by operation of the biological processes of the subject that directs the therapeutic agent to the skin cells in the area of the elamipretide injection site (and elsewhere in the body of the subject).

In some embodiments, the method comprises contacting the elamipretide injection site or intended injection site with an effective amount of a flavonoid, a coumarin, a phenol or a terpenoid as the inhibitor of mast cell degranulation. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

In some embodiments, the elamipretide injection site or intended injection site is contacted with the flavonoid, coumarin, phenol or terpenoid prior to administration of elamipretide, or a pharmaceutically acceptable salt thereof. In some embodiments, the elamipretide injection site or intended injection site is contacted with the flavonoid, coumarin, phenol or terpenoid after administration of elamipretide, or a pharmaceutically acceptable salt thereof. In some embodiments, the elamipretide injection site or intended injection site is contacted with the flavonoid, coumarin, phenol or terpenoid simultaneously with administration of elamipretide, or a pharmaceutically acceptable salt thereof.

In some embodiments, the inhibitor of mast cell degranulation is mometasone furoate, tacrolimus, quercetin, and/or diphenhydramine. In some embodiments, the inhibitor is ice. Thus, in some embodiments, the method comprises contacting the elamipretide injection site or intended injection site with mometasone furoate, tacrolimus, quercetin, diphenhydramine and/or ice. In some embodiments, the elamipretide injection site or intended injection site is contacted with mometasone furoate, tacrolimus, quercetin, and/or diphenhydramine and/or ice prior to administration of elamipretide, or a pharmaceutically acceptable salt thereof. In some embodiments, the elamipretide injection site or intended injection site is contacted with mometasone furoate, tacrolimus, quercetin, and/or diphenhydramine and/or ice after administration of elamipretide, or a pharmaceutically acceptable salt thereof. In some embodiments, the elamipretide injection site or intended injection site is contacted with the mometasone furoate, tacrolimus, quercetin, and/or diphenhydramine and/or ice simultaneously with administration of elamipretide, or a pharmaceutically acceptable salt thereof.

Thus, in some embodiments, mometasone furoate ointment is applied to the elamipretide injection site or intended injection site to thereby contact the elamipretide injection site with mometasone furoate. In some embodiments, tacrolimus ointment or quercetin ointment is applied to the elamipretide injection site or intended injection site to thereby contact the elamipretide injection site with tacrolimus or quercetin. In some embodiments, diphenhydramine or quercetin is administered systemically, optionally via oral administration, to the subject to thereby contact the elamipretide injection site or intended injection site with diphenhydramine or quercetin. In some embodiments, ice is applied to the elamipretide injection site or intended injection site to thereby contact the elamipretide injection site with the ice.

In some embodiments of the method, the elamipretide injection site or intended injection site is contacted with mometasone furoate ointment. In some embodiments of the method, the elamipretide injection site or intended injection site is contacted with tacrolimus ointment. In some embodiments of the method, the elamipretide injection site or intended injection site is contacted with quercetin, either as a topical ointment or cream or indirectly by oral administration of the quercetin. In some embodiments of the method, the elamipretide injection site or intended injection site is contacted with tacrolimus or quercetin. In some embodiments of the method, the elamipretide injection site or intended injection site is contacted with diphenhydramine, often indirectly/systemically by oral administration of the diphenhydramine. In some embodiments of the method, the elamipretide injection site or intended injection site is contacted with quercetin, indirectly/systemically by oral administration of the quercetin. In some embodiments of the method, the elamipretide injection site or intended injection site is contacted with diphenhydramine or quercetin, indirectly/systemically by oral administration of the diphenhydramine or quercetin. In some embodiments of the method, ice is applied to the elamipretide injection site or intended injection site.

In some embodiments of the aforementioned methods, about 5 mg to about 80 mg of elamipretide, or a pharmaceutically acceptable salt thereof, is subcutaneously administered to the injection site of the subject. In some embodiments of the aforementioned methods, about 10 mg to about 60 mg of elamipretide, or a pharmaceutically acceptable salt thereof, is subcutaneously administered to the injection site of the subject. In some embodiments of the aforementioned methods, about 20 mg to about 60 mg of elamipretide, or a pharmaceutically acceptable salt thereof, is subcutaneously administered to the injection site of the subject. In some embodiments of the aforementioned methods, about 20 mg of elamipretide, or a pharmaceutically acceptable salt thereof, is subcutaneously administered to the injection site of the subject. In some embodiments of the aforementioned methods, about 40 mg of elamipretide, or a pharmaceutically acceptable salt thereof, is subcutaneously administered to the injection site of the subject. In some embodiments of the aforementioned methods, about 60 mg of elamipretide, or a pharmaceutically acceptable salt thereof, is subcutaneously administered to the injection site of the subject. In some embodiments of the aforementioned methods, 60 mg of elamipretide, or a pharmaceutically acceptable salt thereof, is subcutaneously administered to the injection site of the subject.

c) Contacting an Injection Site with a Therapeutic Agent

In one aspect, the present disclosure provides a method for treating, preventing, ameliorating, inhibiting or delaying the onset of injection site reactions associated with subcutaneous injections of elamipretide, or a pharmaceutically acceptable salt thereof, comprising contacting an elamipretide injection site or intended injection site with an effective amount of mometasone furoate, tacrolimus, quercetin, diphenhydramine and/or ice. In some embodiments, contacting the elamipretide injection site or intended injection site with mometasone furoate, tacrolimus, quercetin, diphenhydramine and/or ice treats, prevents, ameliorates, inhibits or delays the onset mast cell degranulation in the subject. In some embodiments, contacting the elamipretide injection site or intended injection site with mometasone furoate, tacrolimus, quercetin, diphenhydramine and/or ice treats the elamipretide injection site to thereby arrest mast cell degranulation and the resulting immune response at the elamipretide injection site of the subject. In some embodiments, contacting the elamipretide injection site or intended injection site with mometasone furoate, tacrolimus, quercetin, diphenhydramine and/or ice prevents mast cell degranulation and the resulting immune response at the elamipretide injection site of the subject. In some embodiments, contacting the elamipretide injection site or intended injection site with mometasone furoate, tacrolimus, quercetin, diphenhydramine and/or ice ameliorates mast cell degranulation and the resulting immune response at the elamipretide injection site of the subject. In some embodiments, contacting the elamipretide injection site or intended injection site with mometasone furoate, tacrolimus, quercetin, diphenhydramine and/or ice inhibits mast cell degranulation and the resulting immune response at the elamipretide injection site of the subject. In some embodiments, contacting the elamipretide injection site or intended injection site with mometasone furoate, tacrolimus, quercetin, diphenhydramine and/or ice delays the onset of the injection site reaction by arresting mast cell degranulation and the resulting immune response at the elamipretide injection site of the subject.

In some embodiments, the elamipretide injection site or intended injection site can be contacted with the mometasone furoate, tacrolimus, quercetin, diphenhydramine and/or ice prior to administration of elamipretide, or a pharmaceutical salt thereof. In some embodiments, the elamipretide injection site or intended injection site can be contacted with the mometasone furoate, tacrolimus, quercetin, diphenhydramine and/or ice after administration of elamipretide, or a pharmaceutical salt thereof. In some embodiments, the elamipretide injection site or intended injection site can be contacted with the mometasone furoate, tacrolimus, quercetin, diphenhydramine and/or ice simultaneously with administration of elamipretide, or a pharmaceutical salt thereof.

Thus, in some embodiments, mometasone furoate ointment is applied to the elamipretide injection site or intended injection site to thereby contact the elamipretide injection site with mometasone furoate. In some embodiments, tacrolimus ointment or quercetin ointment is applied to the elamipretide injection site or intended injection site to thereby contact the elamipretide injection site with tacrolimus or quercetin. In some embodiments, diphenhydramine or quercetin is administered systemically, optionally via oral administration, to the subject to thereby contact the elamipretide injection site or intended injection site with diphenhydramine or quercetin. In some embodiments, ice is applied to the elamipretide injection site or intended injection site to thereby contact the elamipretide injection site with the ice.

In some embodiments, contacting the elamipretide injection site or intended injection site with the mometasone furoate, tacrolimus, quercetin, diphenhydramine and/or ice can be can be direct (e.g., applied to the area of the skin where the injection is/was administered) or indirect (e.g. where the inhibitor or mast cell degranulation is administered systematically (e.g., oral or nasal administration) and therefore is applied to the elamipretide injection site or intended injection site by operation of the biological processes of the subject that directs the therapeutic agent to the skin cells in the area of the elamipretide injection site (and elsewhere in the body of the subject).

In some embodiments of the method, the elamipretide injection site or intended injection site is contacted with mometasone furoate ointment. In some embodiments of the method, the elamipretide injection site or intended injection site is contacted with tacrolimus ointment. In some embodiments of the method, the elamipretide injection site or intended injection site is contacted with quercetin, either as a topical ointment or cream or indirectly by oral administration of the quercetin. In some embodiments of the method, the elamipretide injection site or intended injection site is contacted with tacrolimus or quercetin. In some embodiments of the method, the elamipretide injection site or intended injection site is contacted with diphenhydramine, often indirectly/systemically by oral administration of the diphenhydramine. In some embodiments of the method, the elamipretide injection site or intended injection site is contacted with quercetin, indirectly/systemically by oral administration of the quercetin. In some embodiments of the method, the elamipretide injection site or intended injection site is contacted with diphenhydramine or quercetin, indirectly/systemically by oral administration of the diphenhydramine or quercetin. In some embodiments of the method, ice is applied to the elamipretide injection site or intended injection site.

In some embodiments of the aforementioned methods, about 5 mg to about 80 mg of elamipretide, or a pharmaceutically acceptable salt thereof, is subcutaneously administered to the injection site of the subject. In some embodiments of the aforementioned methods, about 10 mg to about 60 mg of elamipretide, or a pharmaceutically acceptable salt thereof, is subcutaneously administered to the injection site of the subject. In some embodiments of the aforementioned methods, about 20 mg to about 60 mg of elamipretide, or a pharmaceutically acceptable salt thereof, is subcutaneously administered to the injection site of the subject. In some embodiments of the aforementioned methods, about 20 mg of elamipretide, or a pharmaceutically acceptable salt thereof, is subcutaneously administered to the injection site of the subject. In some embodiments of the aforementioned methods, about 40 mg of elamipretide, or a pharmaceutically acceptable salt thereof, is subcutaneously administered to the injection site of the subject. In some embodiments of the aforementioned methods, about 60 mg of elamipretide, or a pharmaceutically acceptable salt thereof, is subcutaneously administered to the injection site of the subject. In some embodiments of the aforementioned methods, 60 mg of elamipretide, or a pharmaceutically acceptable salt thereof, is subcutaneously administered to the injection site of the subject.

These doses of elamipretide, or a pharmaceutically acceptable salt thereof, according to any of the methods described in sections III (a) to III(c), above, can be administered once daily to the subject for as long as administration results in beneficial effects on the subject with respect to the disease, disorder or condition being addressed. Alternatively, lesser amounts can be administered several times per day so that that total daily dose is the same as with a single daily dose referred to above.

### d) Method for Treating Mitochondrial Dysfunction

In one aspect, the present disclosure provides a method comprising: (a) subcutaneously administering an effective amount of elamipretide, or a pharmaceutically acceptable salt thereof, to a subject; and (b) administering to said subject an effective amount of an inhibitor of a MRGPRX2 receptor and/or inhibitor of mast cell degranulation, wherein steps (a) and (b) can be performed in either order or simultaneously. In some embodiments, step (a) is performed prior to performing step (b). In some embodiments, step (b) is performed prior to performing step (a). In some embodiments, step (a) and step (b) are performed simultaneously or substantially simultaneously. In some embodiments, the subject is administered an inhibitor of a MRGPRX2 receptor. In some embodiments, the subject is administered an inhibitor of mast cell degranulation. In some embodiment of the foregoing methods, mometasone furoate, tacrolimus, quercetin, diphenhydramine and/or ice is/are administered to the subject. In some embodiments, the subject is human.

In practice of the aforementioned methods, generally from 20 to 80 mg, from 30 to 70 mg or from 40 to 60 mg of elamipretide, or a pharmaceutically acceptable salt thereof, can be subcutaneously administered to the subject. In some embodiments of the aforementioned methods, about 20 mg of elamipretide, or a pharmaceutically acceptable salt thereof, can be subcutaneously administered to the subject. In some embodiments of the aforementioned methods, about 30 mg of elamipretide, or a pharmaceutically acceptable salt thereof, can be subcutaneously administered to the subject. In some embodiments of the aforementioned methods, about 40 mg of elamipretide, or a pharmaceutically acceptable salt thereof, can be subcutaneously administered to the subject. In some embodiments of the aforementioned methods, about 50 mg of elamipretide, or a pharmaceutically acceptable salt thereof, can be subcutaneously administered to the subject. In some embodiments of the aforementioned methods, about 60 mg of elamipretide, or a pharmaceutically acceptable salt thereof, can be subcutaneously administered to the subject. In some embodiments of the aforementioned methods, 60 mg of elamipretide, or a pharmaceutically acceptable salt thereof, can be subcutaneously administered to the subject. In some embodiments of the aforementioned methods, about 70 mg of elamipretide, or a pharmaceutically acceptable salt thereof, can be subcutaneously administered to the subject. In some embodiments of the aforementioned methods, about 80 mg of elamipretide, or a pharmaceutically acceptable salt thereof can be subcutaneously to the subject.

These doses of elamipretide, or a pharmaceutically acceptable salt thereof, can be administered once daily to the subject for as long as administration results in beneficial effects on the subject with respect to the disease, disorder or condition being addressed. Alternatively, lesser amounts can be administered several times per day so that that total daily dose is the same as with a single daily dose referred to above.

### e) Method for 60 mg Dosing of Elamipretide

In one aspect, the present disclosure provides a method comprising subcutaneously administering a 60 mg dose of elamipretide, or a pharmaceutically acceptable salt thereof, to a human subject in need thereof to thereby treat, prevent, ameliorate, inhibit or delay the onset of a disease, disorder or condition associated with mitochondrial dysfunction. In some embodiment, the foregoing method can further comprise administering to the subject an inhibitor of a MRGPRX2 receptor. In some embodiment, the foregoing method can further comprise administering to the subject an inhibitor of mast cell degranulation. In some embodiment of the foregoing methods, mometasone furoate, tacrolimus, quercetin, diphenhydramine and/or ice is/are administered to the subject.

In practice of any of the aforementioned methods disclosed in sections III(a) to III(d), above, in some embodiments, the subject has been diagnosed with Barth syndrome (BTHS).

In practice of any of the aforementioned methods disclosed in sections III(a) to III(d), above, in some embodiments, the subject has been diagnosed with Friedreich's ataxia (FA).

In practice of any of the aforementioned methods disclosed in sections III(a) to III(d), above, in some embodiments, the subject has been diagnosed with age-related macular degeneration (AMD).

In practice of any of the aforementioned methods, in some embodiments, the subject has been diagnosed with macular edema, macular degeneration, glaucoma, geographic atrophy, diabetic retinopathy, or retinitis pigmentosa.

In practice of any of the aforementioned methods disclosed in sections III(a) to III(d), above, in some embodiments, the subject has been diagnosed with primary mitochondrial myopathy.

In practice of any of the aforementioned methods disclosed in sections III(a) to III(d), above, in some embodiments, the subject has been diagnosed with heart failure.

In practice of any of the aforementioned methods disclosed in sections III(a) to III(d), above, in some embodiments, the subject has been diagnosed with hypertrophic cardiomyopathy.

In practice of any of the aforementioned methods disclosed in sections III(a) to III(d), above, in some embodiments, the subject has been diagnosed with muscular dystrophy (MD); including Duchene Muscular Dystrophy (DMD and Becker Muscular Dystrophy (BMD)).

In practice of any of the aforementioned methods disclosed in sections III(a) to III(d), above, in some embodiments, the subject has been diagnosed with primary mitochondrial disease resulting from nuclear DNA mutations (nPMD).

In practice of any of the aforementioned methods disclosed in sections III(a) to III(d), above, in some embodiments, the subject has been diagnosed with glaucoma.

In practice of any of the aforementioned methods disclosed in sections III(a) to III(d), above, in some embodiments, the subject has been diagnosed with geographic atrophy.

In practice of any of the aforementioned methods disclosed in sections III(a) to III(d), above, in some embodiments, the subject has been diagnosed with diabetic retinopathy.

In practice of any of the aforementioned methods disclosed in sections III(a) to III(d), above, in some embodiments, the subject has been diagnosed with retinitis pigmentosa.

### EXAMPLES

The present technology is further illustrated by the following examples, which should not be construed as limiting in any way.

### Example 1—Clinical Study to Evaluate Injection Site Reactions Associated with the Administration of Elamipretide

### Aims of the Study:

Efficacy and safety data from prior human clinical trials support administration of 60 mg subcutaneous (SQ) elamipretide (as the tris-HCl salt) as the appropriate long-term, daily dose. The aim of the present study was to evaluate the efficacy of interventions used to mitigate injection site reactions, and further understand the pharmacokinetics (PK) and safety of 60 mg SQ elamipretide administration in healthy human subjects. Such data and information could be used to inform future clinical studies in subjects suffering from disease, disorders or conditions for which elamipretide is an effective intervention.

### Methods:

### Summary:

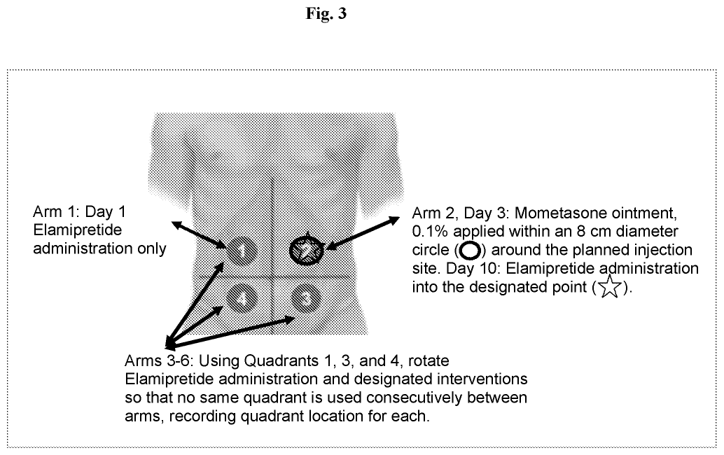

This Phase 1, open-label, 6-arm crossover study included 10 healthy subjects ≥18 and ≤65 years of age.
All subjects received 60 mg of SQ elamipretide to alternating quadrants of the abdomen on each of 6 occasions (FIG. 3), with or without the interventions listed in Table 2.
For each treatment arm, ISR clinical and self-assessments were performed pre-elamipretide administration and at 0.5, 1, 2, 4, 6, 12, 24, and 48 hours after each elamipretide dose.
Blood samples for plasma concentrations of elamipretide and its metabolites were also collected pre-dose and at 0.25, 0.5, 1, 2, 4, and 6 hours post-elamipretide dose.
Primary endpoints consisted of comparisons of the grading from the clinical assessments and the self-assessments of each ISR parameter following administration of elamipretide with each separate intervention versus that of elamipretide alone.
Secondary endpoints consisted of comparison of various pharmacokinetic (PK) parameters and adverse events following administration of elamipretide with each separate intervention versus that of elamipretide alone.

TABLE 2
Summary of the Interventions Administered
and Their Mechanisms of Action (MOA)

| Arm | Intervention | Class/MoA |
|---|---|---|
| 1 | Elamipretide only | Control |
| 2 | Mometasone furoate, 0.1% ointment, | Topical Steroid + |
| | applied under a hydrocolloid occlusive | Occlusive Dressing for |
| | dressing for 7 days prior to elamipretide | Mast Cell Degranulation |
| | administration (Day 10) | |
| 3 | Ice injection site 5 minutes pre- and | Vasoconstriction |
| | post-elamipretide administration | |
| 4 | Tacrolimus, 0.1% cream, applied 15 | Calcineurin Inhibitor |
| | minutes pre-elamipretide administration | Immunosuppressant |
| 5 | Doxepin, 5% cream, applied 15 minutes | Tricyclic Antidepressant: |
| | pre-elamipretide administration | used topically to alleviate |
| | | itching, likely via |
| | | histamine blockade |
| 6 | Diphenhydramine, 50 mg per os (po; by | H1 Antihistamine |
| | mouth), taken 2 hours pre-elamipretide | |
| | administration | |

### Study Design:

After the screening period (up to 28 days) in this phase 1, open-label, 4-week, 6-part, crossover study, all eligible subjects received 60 mg (0.75 mL) of SC elamipretide to alternating quadrants of the abdomen on each of 6 occasions, with or without potential ISR interventions. The first treatment arm (Arm 1) commenced on Day 1 with administration of elamipretide only, followed by the remaining treatment arms (Arms 2-6) with the following potential mitigations: mometasone furoate, 0.1% ointment, applied once under a hydrocolloid occlusive dressing (DuoDERM Extra Thin) 7 days prior to elamipretide administration (Arm 2); ice application at the elamipretide injection site 5 minutes pre- and post-drug administration (Arm 3); tacrolimus 0.1% ointment applied 15 minutes pre-drug dosing (Arm 4); doxepin 5% cream applied 15 minutes pre-elamipretide administration (Arm 5); and diphenhydramine 50 mg orally taken 2 hours prior to administration (Arm 6). For each treatment arm, blood samples for plasma concentrations of elamipretide and its metabolites were collected pre-dose and at 0.25, 0.5, 1, 2, 4, and 6 hours post-elamipretide dose. Vital signs were recorded pre-dose and at 0.5 and 6 hours post-elamipretide administration. ISR clinical and self-assessments were performed pre-elamipretide administration and at 0.5, 1, 2, 4, 6, 12, 24, and 48 hours after each elamipretide dose. Injection sites were photographed at 0.5, 1, 2, 4, and 12 hours post-elamipretide dose.

This study was conducted in accordance with international ethics guidelines, including the Declaration of Helsinki, Council for International Organizations of Medical Sciences International Ethical Guidelines, ICH GCP guidelines, and all applicable laws and regulations. The protocol was approved by the institutional review boards and all participants provided written informed consent prior to beginning the trial.

### Participants:

Healthy subjects ≥18 and 65 years of age with body mass index ≥18.5 and ≤32.0 kg/m² and body weight ≤120 kg were enrolled. Participants had to be willing and able to provide consent and adhere to trial requirements. All participants of childbearing potential were on acceptable birth control. Exclusion criteria included history of clinical hypersensitivity or allergy to any pharmaceutical agent; chronically administered antihistamines or corticosteroids within the last 28 days; and significant mental illness. Tattoos, scarring, or other abdominal skin characteristics which could confound assessment of ISRs were also considered exclusion criteria.

### Study Assessments:

The primary endpoints of this study were comprised of the following efficacy measures: clinical assessments and subject self-assessments. Specifically, the primary endpoints consisted of comparisons of the grading of each ISR parameter following administration of elamipretide with each separate intervention versus the grading of each ISR parameter following administration of elamipretide alone. For clinical assessments, the clinical staff used a standard procedure adapted from the Division of Aids Table for Grading the Severity of Adult and Pediatric Adverse Events to score pain, erythema, induration, and pruritus using a 4-point scale based on severity (0=None, 1=Mild, 2=Moderate, 3=Severe, 4=Potentially Life Threatening). The self-assessments were based on a questionnaire to determine how bothered the patient was following each injection of elamipretide and included the following parameters: pain, burning sensation, cold sensation, itching, redness, swelling, and bruising.

The secondary endpoints of this study consisted of PK and safety assessments. After measuring absorption and distribution PK of elamipretide and its metabolites (M1 and M2), comparisons of each Tₘₐₓ, Cₘₐₓ, and AUC₀₋₆ following administration of elamipretide with each separate intervention versus Tₘₐₓ, Cₘₐₓ, and AUC₀₋₆ following administration of elamipretide alone were performed. Safety assessments consisted of comparison of treatment emergent adverse events (TEAEs) reported following administration of elamipretide with each separate intervention versus TEAEs reported following administration of elamipretide alone. Safety measurements included TEAEs, clinical laboratory tests (e.g., serum chemistry and hematology), 12-lead ECGs, physical exams, and vital signs.

### Statistical Analysis:

All statistical analyses were performed using SAS GRID Linux/SAS Studio (Version 9.4, or higher). PK parameters for elamipretide and its metabolites, M1 and M2, were summarized using descriptive statistics. Comparisons between treatments (elamipretide with each separate intervention versus elamipretide alone) were evaluated by an analysis of the log-transformed PK parameters, Cₘₐₓ and AUC₀₋₆, by performing an analysis of variance (ANOVA) with treatment effects. From these analyses, least square (LS) mean (LSM), LS treatment differences, and 90% confidence interval (CI) for the treatment differences on log-scale were obtained. The results were transformed back to the original scale by exponentiation to provide treatment geometric LSM, point estimates of the geometric test (elamipretide alone)/reference (elamipretide with each separate intervention) LSM ratios, 90% CI for these ratios, and p-value. Chi-square test or Fisher exact test were used to examine the differences in ISR responses between treatment arms at each timepoint.

### Results:

### Summary:

In both the clinical and self-assessments, when compared to Arm 1 (no intervention), ISRs were most reduced with mometasone furoate, ice, tacrolimus, and diphenhydramine (Tables 3 and 4).
In comparison to Arm 1 (no intervention), in Arm 3 (ice application) a 23.5% decrease in Cₘₐₓ and a 12.1% decrease in AUC₀₋₆ was observed, as well as an increase in Tₘₐₓ from 0.5 hours to 1 hours.
   These differences were statistically significant.
   There was no statistical difference between the PK parameters of Arm 1 and the remaining arms of the study.
Across all treatment arms, Treatment Emergent Adverse Events (TEAEs) reported were mild to moderate with the most common being somnolence in the diphenhydramine arm.

TABLE 3
Summary of Clinically Assessed ISRs Across All Timepoints in All Participants (n = 80)

| | Number (%) of Reported Reactions in Elamipretide Treated Subjects | | | | | |
|---|---|---|---|---|---|---|
| | Intervention | | | | | |
| Injection | No | Mometasone | | | | |
| site | intervention | furoate | Ice | Tacrolimus | Doxepin | Diphenhydramine |
| reaction | (Arm 1) | (Arm 2) | (Arm 3) | (Arm 4) | (Arm 5) | (Arm 6) |
| Pain or | 9 | 7 | 7 | 3 | 10 | 2 |
| tenderness | (11.3) | (8.8) | (8.8) | (3.8) | (12.5) | (2.5) |
| Erythema | 21 | 30 | 30 | 25 | 22 | 21 |
| or redness | (26.3) | (37.5) | (37.5) | (31.3) | (27.5) | (26.3) |
| Induration | 16 | 8 | 25 | 18 | 17 | 5 |
| or swelling | (20.0) | (10.0) | (31.3) | (22.5) | (21.3) | (6.3) |
| Pruritis | 14 | 2 | 4 | 12 | 10 | 8 |
| | (17.5) | (2.5) | (5.0) | (15.0) | (12.5) | (10.0) |

TABLE 4
Summary of Self-Assessed ISRs Across All Timepoints in All Participants (n = 80)

| | Number (%) of Reported Reactions in Elamipretide Treated Subjects | | | | | |
|---|---|---|---|---|---|---|
| | Intervention | | | | | |
| Injection | No | Mometasone | | | | |
| site | intervention | furoate | Ice | Tacrolimus | Doxepin | Diphenhydramine |
| reaction | (Arm 1) | (Arm 2) | (Arm 3) | (Arm 4) | (Arm 5) | (Arm 6) |
| Pain | 11 | 7 | 5 | 3 | 7 | 2 |
| | (13.8) | (8.8) | (6.3) | (3.8) | (8.8) | (2.5) |
| Burning | 13 | 7 | 3 | 1 | 6 | 1 |
| sensation | (16.3) | (8.8) | (3.8) | (1.3) | (7.5) | (1.3) |
| Cold | 0.0 | 0.0 | 1 | 0.0 | 0.0 | 0.0 |
| Sensation | | | (1.3) | | | |
| Itching | 16 | 5 | 4 | 12 | 10 | 9 |
| | (20.0) | (6.3) | (5.0) | (15.0) | (12.5) | (11.3) |
| Redness | 24 | 33 | 19 | 20 | 28 | 20 |
| | (30.0) | (41.3) | (23.8) | (25.0) | (35.0) | (25.0) |
| Swelling | 18 | 9 | 15 | 15 | 17 | 5 |
| | (22.5) | (11.3) | (18.8) | (18.8) | (21.3) | (6.3) |
| Bruising | 5 | 1 | 10 | 8 | 0 | 1 |
| | (6.3) | (1.3) | (12.5) | (10.0) | (0.0) | (1.3) |

### Participants:

Out of the 13 subjects screened, 10 individuals underwent treatment and completed the study. Of the 10 participants, 50% were male and the mean age was 40.7 (±5.9) years. Mean weight was 75.31 (±11.35) kg; mean height was 167.98 (±9.25) cm; and mean BMI was 26.58 (±2.92) kg/m². All participants were White with 90% classified as Fitzpatrick skin type III—darker white (tans after initial burn) and 10% classified as Fitzpatrick skin type IV—light brown (burns minimally, tans easily).

### Efficacy Outcomes:

Analysis of the clinical assessments of the ISRs provided very few statistically significant variables in this small sample size. As shown in Table 1, Arm 2 (mometasone furoate) demonstrated significantly reduced incidence of induration at 0.5 hours post-elamipretide dose (p=0.0031). Trends to significance in reduction of induration at 1 hour post-dose (p=0.0736) and pruritus at 0.5 hours post-dose (p=0.0573) and were also observed in Arm 2 (Tables 1 and 2). Treatment Arm 6 (diphenhydramine) showed significantly decreased incidence of induration at 1 hour post-elamipretide dose (p=0.0198) and a trend to significance in reduction of induration at 0.5 hours post-dose (p=0.0698; Table 1).

Similarly, very few statistically significant variables were returned in the subject ISR self-assessments. As shown in Table 3, Arm 3 (ice) demonstrated reduced incidence of pain at 0.5 hours post-elamipretide dose (p=0.0325). A trend to significance in reduction of itching at 0.5 hours after elamipretide dosing (p=0.0573) was also observed (Table 4). No further p-values of <0.08 were observed in any of the intervention arms at any timepoints.

The elamipretide injection site photographs collected aligned with the signs and symptoms of ISRs commonly described following administration of elamipretide by SC injection. In addition, the photographs supported the clinical assessments of the ISRs that were conducted at the same timepoints. Overall, the ISRs captured on the photographs were resolving at 4 hours post-elamipretide dose with the exception of bruising, which appeared to form after the 12-hour post-dose timepoint in those affected. In Arm 2 (mometasone furoate), the photographs aided in deciphering erythema grading in subjects where the pattern of redness appeared to be related to the use of the occlusive dressing and not the injection.

### Pharmacokinetics:

In all study arms where elamipretide was administered with a topical or systemic drug, the AUC₀₋₆ was not significantly different to that of elamipretide given alone. Arm 3 (ice) demonstrated a statistically significant impact on the Cₘₐₓ and AUC₀₋₆ of elamipretide when compared to Arm 1 (elamipretide alone). Specifically, the Cₘₐₓ of elamipretide was reduced by approximately 23% (p=0.0003) and the AUC₀₋₆ of elamipretide was reduced by approximately 12% (p<0.0001). Through analysis of the PK data on the metabolites of elamipretide, Arm 3 (ice) showed a statistically significant reduction on the Cₘₐₓ and AUC₀₋₆ of M1 and on the AUC₀₋₆ of M2.

### Safety:

With the exception of data relating to ISRs, few TEAEs were reported in this study. The only TEAE observed in more than 1 subject was that of somnolence in Arm 6 (diphenhydramine) where 5 subjects (50%) reported mild somnolence. There were no observed differences between any treatment arm in vital signs, ECG parameters.

### Discussion:

This study sought to evaluate the efficacy of interventions used to mitigate ISRs, while analyzing the PK and safety, of 60 mg SC elamipretide administration in healthy subjects. Overall, mometasone furoate appeared to favorably mitigate the pseudo-allergic reactions observed following SC injection of elamipretide, specifically reducing pruritus and induration. Ice application ameliorated early injection site pain and itching, while diphenhydramine demonstrated potential in reducing induration. Tacrolimus and doxepin demonstrated little impact on reported ISRs compared to elamipretide alone and are not likely to be recommended for ISR mitigation. No significant changes in elamipretide plasma exposure were reported in any of the treatment arms, with the exception of Arm 3 (ice application), when compared to elamipretide administered alone.

Although ice application and diphenhydramine showed some reduction in ISR signs and symptoms, mometasone furoate revealed the most promise. The reduction in pruritus and induration demonstrated by mometasone furoate is an excellent outcome and enabled patients to be more comfortable with the elamipretide treatment. In addition, mometasone furoate may further increase elamipretide tolerability by reducing scratching and subsequent scratching-related skin damage. In some subjects, the hydrocolloid occlusive dressing applied over the mometasone furoate ointment resulted in redness of the covered area (i.e., the injection site area), confounding the ability to identify erythema due to ISRs. The capture of ISR photographs proved useful in deciphering erythema grading in subjects where the pattern of redness appeared to be related to the use of the occlusive dressing. However, it is possible that erythema as an ISR was over reported in Arm 2 (mometasone furoate) of this study.

Despite ice application and diphenhydramine reducing some ISR signs and symptoms, both interventions presented undesirable effects. Specifically, ice application improved early injection site pain and itching, but this simple intervention caused vasoconstriction and expectedly reduced the Cₘₐₓ and AUC₀₋₆ of elamipretide and its metabolites (M1 and M2). Neither M1 nor M2 are biologically active and therefore the potential reduction in plasma concentrations of these metabolites is not anticipated to be impactful. However, while the potential ramification on efficacy of the disruption in absorption and distribution of the active parent drug is unclear, lowering plasma exposure of elamipretide is not desirable, making ice application a less appropriate mitigation option. Similarly, diphenhydramine showed some potential in reducing induration, but clinically significant incidence (50%) of mild somnolence was reported in this treatment arm. Diphenhydramine is not likely to be recommended for ISR mitigation given its little impact on ISR symptoms and the prevalence of somnolence, but the drug could potentially be useful if elamipretide is dosed daily at nighttime.

To further improve ISRs following elamipretide administration, it is important to understand the mechanism underlying the reactions. SC elamipretide induces immediate hypersensitivity reactions in drug naïve individuals, implying IgE-independent mechanisms. In vitro assays demonstrated that elamipretide activates the Mas-related G-protein coupled receptor member X2 (MRGPRX2), a receptor expressed almost exclusively by a subset of mast cells that populate connective tissues like the skin (Motakis et al. 2014; Tatemoto et al. 2006). MRGPRX2 is known to be involved in drug-induced pseudo-allergic reactions and commonly activated by cationic peptides and small molecules (McNeil 2021). Specifically, elamipretide mobilizes calcium in MRGPRX2-expressing cells in a dose-dependent manner; however, stimulation of MRGPRX2-expressing cells with M1 and M2 peptides does not result in significant calcium mobilization. Itch produced in humans by injection of MRGPRX2 agonists can be blocked by antihistamines (Hasbak et al. 2006; Steinhoff et al. 2003), but development of inhibitors is still in its infancy with no candidates in clinical trial yet (McNeil 2021). While the topical steroid mometasone furoate reduced pruritus and induration following SC elamipretide in this study, the anti-inflammatory mechanism is unclear and is thought to act by inhibiting the arachidonic acid pathway (US Food and Drug Administration). In addition to anti-inflammatory effects, topical steroids, such as mometasone, possess anti-mitotic and immunosuppressive effects (Gabros et al. 2021), which may have played a role in mitigating ISR signs in this study. For the purpose of ameliorating the pseudo-allergic reactions following SC elamipretide, development of high-affinity MRGPRX2 antagonists might be beneficial.

Overall, the data collected in this study support prior findings that SC elamipretide is generally safe and well-tolerated (Thompson et al. 2021; Karaa et al. 2020; Mettu et al. 2022). With the exception of data relating to ISRs, very few TEAEs were identified in this study. The only TEAE seen in more than 1 subject was that of somnolence in the diphenhydramine treatment arm. Given that somnolence is a well-known side effect of diphenhydramine (Sicari and Zabbo 2021), this adverse event was considered likely related to diphenhydramine and not to elamipretide treatment. A limitation of this study was the small sample size, however the sample size of 10 subjects was based on precedent set by prior Phase 1 studies of similar nature and design.

### Conclusions:

Subcutaneous (SQ) elamipretide administration is generally safe and well-tolerated with ISRs being the most commonly reported adverse event.
Improvement in elamipretide ISRs (particularly erythema and pruritis) was noted in subjects treated with mometasone furoate ointment, tacrolimus ointment, diphenhydramine (oral), and ice.
   All of these treatments are readily available and generally well-tolerated.
   Cmax and AUC0-6 were significantly reduced in those treated with ice application. As such, ice would be least preferred over other interventions.
The hydrocolloid occlusive dressing applied over the mometasone furoate ointment (Arm 2) often resulted in redness of the covered area (i.e., the elamipretide injection site area), confounding the ability to evaluate erythema due to ISRs.
For patients who experience ISRs with SQ elamipretide administration, it is reasonable to recommend mometasone furoate ointment, tacrolimus ointment, or diphenhydramine as a front line intervention to mitigate ISRs.
Applying ice to the elamipretide injection site should not be routinely utilized due to the decrease in elamipretide exposure and should only be considered as a last line recommendation.

### Example 2—Injection Site Reactions Induced by Elamipretide are Mediated by Activation of Mast Cells Through the G Protein-Coupled Receptor MRGPRX2

### Summary:

This example was performed to assess whether or not local injection site reactions associated with subcutaneous elamipretide administration might, at least in part, be mediated by the G protein-coupled receptor MRGPRX2 and involve activation of mast cells in the skin of the human subject or mouse to thereby induce the observed inflammatory response.

### Experimental Design

### EC50 Value for MRGPRX2

A clonal HEK293 cell line stably expressing human MRGPRX2 and Galpha15 was used for the assay, which was performed using a fluorescent plate reader. Cells were plated at 40,000 cells per well in a glass-bottom 96 well plate 20 hours before the assay. Cells were loaded with N-[4-[6-[(Acetyloxy)methoxy]-2,7-difluoro-3-oxo-3H-xanthen-9-yl]-2-[2-[2-[bis[2-[(acetyloxy)methoxy]-2-oxoethyl]amino]-5-methylphenoxy]ethoxy]phenyl]-N-[2-[(acetyloxy)methoxy]-2-oxoethyl]glycine (acetyloxy)methyl ester (Fluo-4 AM) for 45 minutes at 37° C. and allowed to rest for 30 minutes before use. Baseline fluorescence was calculated as the average of a 30 second read, with data points taken at 5 second intervals. Response was defined as the maximum signal within 90 seconds after addition of elamipretide, subtracted by the baseline fluorescence signal. Concentrations were tested in duplicate and the assay was run 6 times. The curve was calculated as a four-parameter non-linear fit with variable slope. Traces were obtained using the same cell line and same Fluo-4 AM loading protocol but were imaged using a fluorescence microscope using a standard fluorescein isothiocyanate (FITC) filter. Fluorescence for different cells was calculated using region of interest selections.

### Mast Cell Activation

Mouse primary peritoneal mast cells were isolated from wild type and Mrgprb2 knockout mice. Mast cells were incubated in Dulbecco's Modified Eagle Medium, supplemented with 10% fetal bovine serum and 100 ng/mL human stem cell factor for 2 hours in wells of a glass-bottom 96 well plate that had been previously coated with fibronectin. Mast cells were then loaded with Fluo-4 AM for 30 minutes at room temperature, washed, and allowed to rest for 30 minutes before use. A confocal fluorescence microscope was used to assess changes in free intracellular calcium levels in each mast cell in a visual field, as measured by changes in Fluo-4 fluorescence using a standard FITC filter, reflecting responsiveness to elamipretide. Cells were identified as responding if the Fluo-4 signal rose by at least 50% for at least 10 seconds, which clearly distinguishes a ligand-induced response from random flickering events.

### Evans Blue Assay of In Vivo Inflammation

Adult mice up to 8 months of age were anesthetized with an intraperitoneal injection of ketamine/xylazine. Fifteen (15) minutes after induction of anesthesia, mice were injected intravenously with 50 μL of 12.5 mg/mL Evans Blue in saline. Five (5) minutes later, 5 μL of 0.5 mg/mL elamipretide (in saline) was administered by injection in one paw and saline was administered in the other paw. Fifteen (15) minutes later, mice were sacrificed by decapitation. Paw tissue was collected, dried for 24 hours at 50° C., and weighed. Evans Blue was extracted by a 24-hour incubation in formamide at 50° C., and the optical density (O.D.) was read at 600 nm (the maximum Evans Blue absorbance) and 740 nm (the minimum specific Evans Blue absorbance) using a spectrophotometer. The value at 740 nm, reflecting Evans Blue-independent O.D., was subtracted from the value at 600 nm to attain the final readout.

### Results:

1.) MRGPRX2 and Mrgprb2 were Activated by Elamipretide Administration

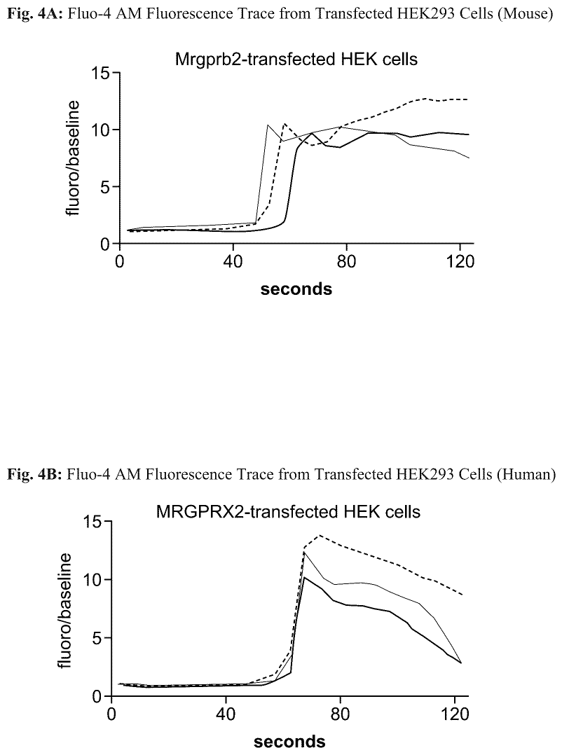

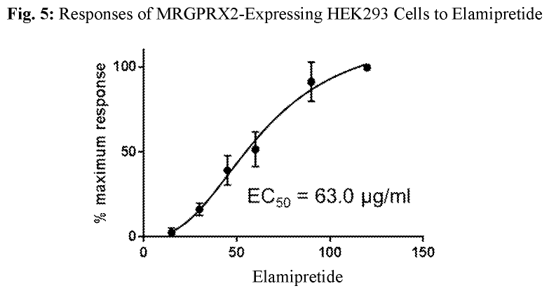

A calcium-based readout of receptor activity was used to determine whether elamipretide activates human MRGPRX2 and its mouse ortholog Mrgprb2. This assay is known to be widely used in drug screening and utilized a cell line stably transfected with the receptor along with Galpha15, a promiscuous G protein that couples to the receptor and induced an increase in intracellular calcium when the receptor was activated. Thus, an increase in calcium levels reflected receptor activation. Calcium levels were detected by imaging Fluo-4 (from Fluo-4 AM), a dye that increases in fluorescence when bound to calcium. Fluo-4 fluorescence increased after elamipretide exposure in HEK293 cells transfected with MRGPRX2 (FIG. 4B) or its mouse ortholog Mrgprb2 (FIG. 4A), but not in untransfected cells (data not shown). Using a fluorescence plate reader, it was determined that the EC50 for MRGPRX2 was 63±13 micrograms/mL (FIG. 5). This is much lower than the typical elamipretide injection concentration of 40 mg/mL and demonstrates that elamipretide almost certainly activated MRGPRX2 in vivo.

2.) Mast Cells were Activated by Elamipretide in an Mrgprb2-Dependent Manner

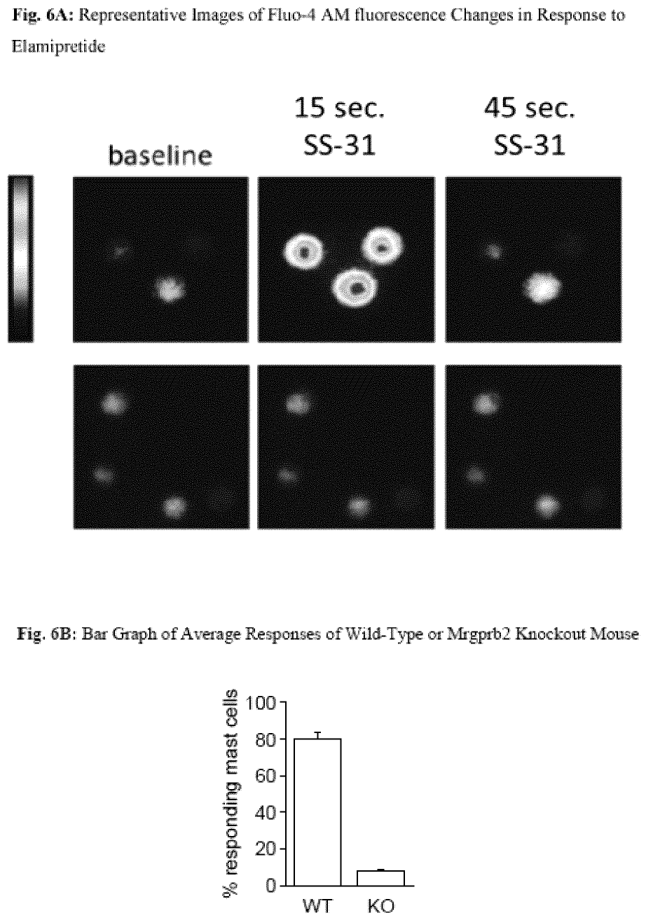

Injection site reactions induced by several classes of drugs were mediated in mice by Mrgprb2-dependent activation of mast cells. Whether elamipretide activated primary mouse connective tissue mast cells was evaluated, and if so, it was evaluated whether this was Mrgprb2-dependent. Mast cell mediator release was dependent on rises in intracellular calcium levels, and compounds that increase intracellular calcium in mast cells induce exocytosis, accordingly a Fluo-4-based calcium detection assay was used to assess mast cell activation. Briefly, primary connective tissue mast cells were purified from mice, loaded with Fluo-4, and changes in fluorescence after elamipretide exposure were imaged. T fluorescence of over 200 individual mast cells, each from wild type and Mrgprb2 knockout mice, were monitored and the percentage of total cells that responded was determined. Robust increases in intracellular calcium in response to elamipretide in most wild-type mast cells were observed, but almost no responses in mast cells from Mrgprb2 knockout mice were observed (FIGS. 6A and 6B).

### 3.) In Vivo Inflammation in Mice is Induced by Elamipretide in an Mrgprb2-Dependent Manner

Finally, it was determined whether elamipretide induced injection site reactions in mice, and if so, whether the injection site reactions were dependent on Mrgprb2. Injection site reactions were characterized by local inflammation and edema. The standard assay in mice to monitor local skin inflammation is the Evans Blue assay. In conditions of local skin inflammation, blood vessels become more permeable and components of blood leak out into the local tissue. Evans Blue is a dye that binds to a protein called albumin in the blood and leaks out with the albumin in inflammatory conditions. In this assay, mice were injected intravenously with Evans Blue and the amount of Evans Blue leakage in a tissue was a readout of the extent of inflammation. A common injection site when assaying inflammation in mice is the footpad because it is thick and relatively compartmentalized, so whatever is injected does not immediately diffuse away. This is preferable to other skin sites in mice, as they are extremely thin, compared to human skin, and injected fluids almost immediately leave the sites of injection. It was found that injection of elamipretide into mouse footpads induced strong Evans Blue labeling, while saline injection induced much less Evans Blue labeling (FIGS. 7A and 7B). The labeling was dependent on the presence of Mrgprb2, as elamipretide induced almost no Evans Blue accumulation in Mrgprb2 knockout mice (FIGS. 7A and 7B). Measurements of Evans Blue accumulation in footpads revealed that accumulation was 80% less in Mrgprb2 knockout mice, demonstrating that, at this concentration (0.5 mg/mL elamipretide), most of the inflammation was dependent on Mrgprb2.

### Conclusion:

Subcutaneous injection of elamipretide (SS-31) was associated with local injection site reactions. Injection site reactions were mediated by the G protein-coupled receptor MRGPRX2, which is specifically expressed by mast cells in the skin and, when activated, induced strong inflammatory reactions. This study determined that MRGPRX2 was activated by elamipretide, and that its EC50 of 63±13 micrograms/mL was ˜600 times lower than the typical elamipretide injection concentration of 40 mg/mL. Elamipretide was also shown to induce strong mast cell activation and local inflammation in mice after subcutaneous injection, both of which were dependent on Mrgprb2, the mouse ortholog of MRGPRX2. Elamipretide also activated the human Mrgprb2 ortholog MRGPRX2 at physiologically relevant concentrations, suggesting that mast cell activation by MRGPRX2 underlies the injection site reactions observed in humans. Based on these results, treatment options that target mast cell activation or the effects of mast cell-derived mediators may significantly ameliorate injection site reactions in humans caused by subcutaneous administration of elamipretide.

### Example 3—Injection Site Reactions Induced by Elamipretide are Mediated by Activation of Mast Cells Through the G Protein-Coupled Receptor MRGPRX2

### Summary:

This study was conducted to demonstrate the feasibility of preparing an in vitro assay to determine whether or not elamipretide and its known metabolites activate the human MRGPRX2 receptor and thereby induce mast cell degranulation as well as provide a possible screening assay to determine the efficacy of potential inhibitors of the activation of the human MRGPRX2 receptor.

### Experimental:

MRGPRX2 expressing and control (non-MRGPRX2 expressing) Chem-1 cells lines were obtained from Discovery Services, EuroFins. Cells were cultured in Dulbecco's Modified Eagle Medium (DMEM) high glucose medium (4.5 g/L) with 10% fetal bovine serum (FBS), non-essential amino acids, and N-2-hydroxyethylpiperazine-N′-2-ethanesulfonic acid (HEPES) buffer (pH 7.0-7.6). Geneticin (G418) was used for selection of MRGPRX2-expressing Chem-1 cells. Cells were maintained at 37° C. at 5% CO₂. Cells were seeded to a glass bottom 96-well plates at a density of 50,000 per well (Cellvis). Cells were loaded with Fluo-4 AM for 30 minutes at 37° C. at a concentration of 2.5 μM. Cells were washed once with Dulbecco's phosphate buffered saline (DPBS). Cells were rested at room temperature for 30 minutes before use and washed with DPBS. Cells were resuspended in 50 μl phosphate buffered saline (PBS) with calcium and magnesium. To stimulate cells, the cells were contacted with 50 μL of 2× drug (elamipretide or leuprolide), or DPBS as a control. Plates were read immediately with a Biotek plate reader. Readings were taken every 6 seconds for 2 minutes.

This assay was designed as a calcium-based readout of receptor activity as used on Example 2. As such, the assay can be used to determine if a test article will elicit an immune response associated with the MRGPRX2 receptor, as well as possibly demonstrating the efficacy of inhibitors of the receptor.

### Results:

With reference to FIG. 8A and FIG. 8B, the results demonstrate that elamipretide exhibits a dose dependent activation of the MRGPRX2 expressing cells (FIG. 8A) but no effect on cells lacking the MRGPRX2 receptor (FIG. 8B). Ionomycin is a positive control that generates a calcium response regardless of the presence or absence of the MRGPRX2 receptor (the parent Chem-1 cells lack the MRGPRX2 receptor) and thereby confirms that the cells of the assay are responsive to calcium flux.

With reference to FIG. 9, the results demonstrate that leuprolide exhibits a dose dependent activation of the MRGPRX2 expressing cells. Leuprolide is an expected activator of the MRGPRX2 receptor and therefore confirms the expected function of the assay when an activator of the MRGPRX2 receptor is present. In this way, the interpretation of the results of FIG. 8A and FIG. 8B are further confirmed.

With reference to FIG. 10A and FIG. 10B, the results indicate that, unlike elamipretide itself, the known M1 and M2 metabolites of elamipretide do not activate MRGPRX2 receptor. This result further confirms that activation of the MRGPRX2 receptor in this assay as seen in FIG. 8A is a function of the presence of elamipretide itself and not its metabolites.

With reference to FIG. 11, the results indicate that quercetin can, in a dose-dependent manner, inhibit the activation of the MRGPRX2 receptor that is caused by the presence of elamipretide. As such, the results suggest that this assay can be used to screen possible inhibitors suitable to treat, prevent, ameliorate, inhibit or delay the onset of injection site reactions caused by subcutaneous injection of elamipretide in a subject.

### Conclusions:

The data in this Example 3 support the following conclusions:
Elamipretide activates the human MRGPRX2 receptor;
Metabolites M1 and M2 of elamipretide do not activate the human MRGPRX2 receptor;
It is possible to inhibit elamipretide-mediated activation of MRGPRX2;
This in vitro assay can be used to screen possible inhibitors of the activation of the human MRGPRX2 receptor; and
Quercetin is an inhibitor of the activation of the human MRGPRX2 receptor.

### REFERENCES

Arkin, M. R. et al. in Assay Guidance Manual (eds G. S. Sittampalam et al.) (2004).
Birk A. V., et al. The mitochondrial-targeted compound SS-31 re-energizes ischemic mitochondria by interacting with cardiolipin. J Am Soc Neprhol. 2013; 24:2360-1261.
Finn D. F. et al. Twenty-first century mast cell stabilizers. British Journal of Pharmacology. 2013; 170: 23-37
Gabros S, Nessel T A, Zito P M. Topical corticosteroids. In: StatPearls. Treasure Island, FL: StatPearls Publishing, 2020.
Hasbak, et al., The Vasorelaxant Effect of Adrenomedullin, Proadrenomedullin N-Terminal 20 Peptide and Amylin in Human Skin, Basic & Clinical Pharmacology & Toxicology, 99, 162-167 (2006).
Karaa A., et al. A randomized crossover trial of elamipretide in adults with primary mitochondrial myopathy. J Cachexia Sarcopenia Muscle. 2020 August; 11(4):909-918.
Karaa A., et al. Randomized dose-escalation trial of elamipretide in adults with primary mitochondrial myopathy. Neurology. 2018; 90(14):e1212-e1221.
Ma, H. T. & Beaven, M. A. Regulators of Ca(2+) signaling in mast cells: potential targets for treatment of mast cell-related diseases? Adv Exp Med Biol 716, 62-90, doi:10.1007/978-1-4419-9533-9_5 (2011).
McNeil, B. D. et al. Identification of a mast-cell-specific receptor crucial for pseudoallergic drug reactions. Nature 519, 237-241, doi:10.1038/nature14022 (2015).
McNeil, et al., MRGPRX2 and Adverse Drug Reactions, Frontiers in Immunology 12:1-13 (2021).
Mettu, et al., Phase 1 Clinical Trial of Elamipretide in Dry Age-Related Macular Degeneration and Noncentral Geographic Atrophy, Ophthalmology Science, 2(1): 1-10 (2022).
Mitchell W., et al. The mitochondria-targeted peptide SS-31 binds lipid bilyers and modulates surface electrostatics as a key component of its mechanism of action: SS-31 modulates lipid bilayer electrostatics. Journal of Biological Chemistry 2020; 295(21):7452-7469.
Motakis, et al., Redefinition of the human mast cell transcriptome by deep-CAGE sequencing, Blood, 123(17): e58-e67 (2014).
Radu, M. & Chernoff, J. An in vivo assay to test blood vessel permeability. J Vis Exp, e50062, doi:10.3791/50062 (2013).
Sabbah H. D., et al. Effects of elamipretide on skeletal muscle in dogs with experimentally induced heart failure. ESC Heart Failure. 2019; 6:328-335.
Sicari, V. & Zabbo, C., Diphenhydramine, In: StatPearls. Treasure Island, FL: StatPearls Publishing, 2022.
Steinhoff, et al., Proteinase-Activated Receptor-2 Mediates Itch: A Novel Pathway for Pruritus in Human Skin, J. Neurosci., 23(15):6176-6180 (2003).
Szeto H. H. & Birk A. V. Serendipity and the discovery of novel compounds that restore mitochondrial plasticity. Clin Pharmacol Ther. 2014; 96:972-683.
Tatemoto, K., et al., Immunoglobulin E-independent activation of mast cell is mediated by Mrg receptors, Biochem Biophys Res Commun. 2006, 349(4):1322-8.
Thompson R. W., et al. A phase 2/3 randomized clinical trial followed by an open-label extension to evaluate the effectiveness of elamipretide in Barth syndrome, a genetic disorder of mitochondrial cardiolipin metabolism. Genet Med. 2021 March; 23(3):471-478.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a nonlimiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. A method for treating, preventing, ameliorating, inhibiting or delaying the onset of injection site reactions associated with subcutaneous injections of elamipretide, or a pharmaceutically acceptable salt thereof, administered to a subject in need thereof, comprising contacting an elamipretide injection site or intended injection site with an effective amount of an inhibitor of a MRGPRX2 receptor.

2. The method of claim 1, wherein contacting the elamipretide injection site or intended injection site with the inhibitor of the MRGPRX2 receptor treats, prevents, ameliorates, inhibits or delays the onset of mast cell degranulation in the subject.

3. The method of claim 1, wherein the method comprises contacting the elamipretide injection site or intended injection site of the subject with an effective amount of a flavonoid, a coumarin, a phenol or a terpenoid as the inhibitor of a MRGPRX2 receptor.

4. The method of claim 3, wherein the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin.

5. The method of claim 3, wherein the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione).

6. The method of claim 3, wherein the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one).

7. The method of claim 3, wherein the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

8. The method claim 3, wherein the elamipretide injection site or intended injection site is contacted with the flavonoid, the coumarin, the phenol or the terpenoid prior to administration of elamipretide, or a pharmaceutically acceptable salt thereof.

9. The method claim 3, wherein the elamipretide injection site or intended injection site is contacted with the flavonoid, the coumarin, the phenol or the terpenoid after administration of elamipretide, or a pharmaceutically acceptable salt thereof.

10. The method of claim 1, wherein the method comprises contacting the elamipretide injection site or intended injection site with an effective amount of mometasone furoate, tacrolimus, quercetin, diphenhydramine and/or ice.

11. The method of claim 10, wherein mometasone furoate ointment is applied to the elamipretide injection site or intended injection site to thereby contact the elamipretide injection site or intended injection site with mometasone furoate.

12. The method of claim 10, wherein tacrolimus ointment or quercetin ointment is applied to the elamipretide injection site or intended injection site to thereby contact the elamipretide injection site or intended injection site with tacrolimus or quercetin.

13. The method of claim 10, wherein diphenhydramine or quercetin is administered systemically, optionally via oral administration, to the subject to thereby contact the elamipretide injection site or intended injection site with diphenhydramine or quercetin.

14. The method of claim 10, wherein ice is applied to the elamipretide injection site or intended injection site to thereby contact the elamipretide injection site or intended injection site with the ice.

15. The method claim 10, wherein the elamipretide injection site or intended injection site is contacted with the mometasone furoate, the tacrolimus, the quercetin, the diphenhydramine and/or the ice prior to administration of elamipretide, or a pharmaceutically acceptable salt thereof.

16. The method of claim 10, wherein the elamipretide injection site or intended injection site is contacted with the mometasone furoate, the tacrolimus, the quercetin, the diphenhydramine and/or the ice after administration of elamipretide, or a pharmaceutically acceptable salt thereof.

17. The method claim 1, wherein about 5 mg to about 60 mg of elamipretide, or a pharmaceutically acceptable salt thereof, is subcutaneously administered to the elamipretide injection site of the subject.

18. The claim 1, wherein 60 mg of elamipretide, or a pharmaceutically acceptable salt thereof, is subcutaneously administered to the elamipretide injection site of the subject.

19. A method for treating, preventing, ameliorating, inhibiting or delaying the onset of injection site reactions associated with subcutaneous injections of elamipretide, or a pharmaceutically acceptable salt thereof, administered to a subject in need thereof, comprising contacting an elamipretide injection site or intended injection site with an effective amount of an inhibitor of mast cell degranulation.

20. The method of claim 19, wherein the method comprises contacting the elamipretide injection site or intended injection site with an effective amount of a flavonoid, a coumarin, a phenol or a terpenoid as the inhibitor of mast cell degranulation.

21. The method of claim 20, wherein the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin.

22. The method of claim 20, wherein the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione).

23. The method of claim 20, wherein the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl)ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one).

24. The method of claim 20, wherein the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9(1aH)-one), sinomenine, indoline (2,3-dihydro-1H-indole) or xestospongin C ([1R-(1R,4aR,11R,12aS,13S,16aS,23R,24aS)]-eicosahydro-5H,17H-1,23:11,13-diethano-2H,14H-[1,11]dioxacycloeicosino[2,3-b:12,13-b1]dipyridine).

25. The method claim 20, wherein the elamipretide injection site or intended injection site is contacted with the flavonoid, the coumarin, the phenol or the terpenoid prior to administration of elamipretide, or a pharmaceutically acceptable salt thereof.

26. The method claim 20, wherein the elamipretide injection site or intended injection site is contacted with the flavonoid, the coumarin, the phenol or the terpenoid after administration of elamipretide, or a pharmaceutically acceptable salt thereof.

27. The method of claim 19, wherein the method comprises contacting the elamipretide injection site or intended injection site with an effective amount of mometasone furoate, tacrolimus, quercetin, diphenhydramine and/or ice.

28. The method of claim 27, wherein mometasone furoate ointment is applied to the injection site or intended injection site to thereby contact the elamipretide injection site with mometasone furoate.

29. The method of claim 27, wherein tacrolimus ointment or quercetin ointment is applied to the injection site or intended injection site to thereby contact the elamipretide injection site with tacrolimus or quercetin.

30. The method of claim 27, wherein diphenhydramine or quercetin is administered systemically, optionally via oral administration, to the subject to thereby contact the elamipretide injection site or intended injection site with diphenhydramine or quercetin.

31. The method of claim 27, wherein ice is applied to the injection site or intended injection site to thereby contact the elamipretide injection site with the ice.

32. The method claim 27, wherein the elamipretide injection site or intended injection site is contacted with the mometasone furoate, the tacrolimus, the quercetin, the diphenhydramine and/or the ice prior to administration of elamipretide, or a pharmaceutically acceptable salt thereof.

33. The method claim 27, wherein the elamipretide injection site or intended injection site is contacted with the mometasone furoate, the tacrolimus, the quercetin, the diphenhydramine and/or the ice after administration of elamipretide, or a pharmaceutically acceptable salt thereof.

34. The method claim 19, wherein about 5 mg to about 60 mg of elamipretide, or a pharmaceutically acceptable salt thereof, is subcutaneously administered to the elamipretide injection site of the subject.

35. The method claim 19, wherein 60 mg of elamipretide, or a pharmaceutically acceptable salt thereof, is subcutaneously administered to the elamipretide injection site of the subject.

36. A method for treating, preventing, ameliorating, inhibiting or delaying the onset of injection site reactions associated with subcutaneous injections of elamipretide, or a pharmaceutically acceptable salt thereof, administered to a subject in need thereof, comprising contacting an elamipretide injection site or intended injection site with a flavonoid, a coumarin, a phenol or a terpenoid.

37.-43. (canceled)

44. The method claim 36, wherein 60 mg of elamipretide, or a pharmaceutically acceptable salt thereof, is subcutaneously administered to the elamipretide injection site of the subject.

45. A method for treating, preventing, ameliorating, inhibiting or delaying the onset of injection site reactions associated with subcutaneous injections of elamipretide, or a pharmaceutically acceptable salt thereof, administered to a subject in need thereof, comprising contacting an elamipretide injection site or intended injection site with mometasone furoate, tacrolimus, quercetin, diphenhydramine and/or ice.

46.-53. (canceled)

54. A method comprising:
a) subcutaneously administering an effective amount of elamipretide, or a pharmaceutically acceptable salt thereof, to a subject; and
b) administering to said subject an effective amount of an inhibitor of a MRGPRX2 receptor and/or inhibitor of mast cell degranulation;
wherein steps a) and b) can be performed in either order or simultaneously.

55. The method of claim 54, wherein step (a) is performed prior to performing step (b).

56. The method of claim 54, wherein step (b) is performed prior to performing step (a).

57. The method of claim 54, wherein step (a) and step (b) are performed simultaneously or substantially simultaneously.

58.-68. (canceled)

69. A method comprising subcutaneously administering a 60 mg dose of elamipretide, or a pharmaceutically acceptable salt thereof, to a human subject in need thereof to thereby treat, prevent, ameliorate, inhibit or delay the onset of a disease, disorder or condition associated with mitochondrial dysfunction.

70. The method of claim 69, further comprising administering to the subject an inhibitor of a MRGPRX2 receptor.

71. The method of claim 69, further comprising administering to the subject an inhibitor of mast cell degranulation.

72.-88. (canceled)

89. The method of claim 1, wherein 40 mg of elamipretide, or a pharmaceutically acceptable salt thereof, is subcutaneously administered to the elamipretide injection site of the subject.

90. The method of claim 19, wherein 40 mg of elamipretide, or a pharmaceutically acceptable salt thereof, is subcutaneously administered to the elamipretide injection site of the subject.

91. The method of claim 36, wherein 40 mg of elamipretide, or a pharmaceutically acceptable salt thereof, is subcutaneously administered to the elamipretide injection site of the subject.
